(19) United States
(12) Patent Application Publication
(10) Pub. No.: US 2016/0022488 A1
(43) Pub. Date: Jan. 28, 2016

(54) INTRAOCULAR LENS FIXATION CORRECTION METHODS AND DEVICES

(71) Applicant: Optic Logik LLC, Bend, OR (US)

(72) Inventors: Jason W. Dimmig, Bend, OR (US); Huckleberry A. Holz, San Francisco, CA (US)

(73) Assignee: Optic Logik LLC, Bend, OR (US)

(21) Appl. No.: 14/773,546

(22) PCT Filed: Mar. 7, 2014
(86) PCT No.: PCT/US2014/021873

§ 371 Date: Sep. 8, 2015

Related U.S. Application Data
(60) Provisional application No. 61/774,792, filed on Mar. 8, 2013.

(51) Int. Cl. A61F 9/00

(52) U.S. Cl. CPC A61F 9/0026

(57) ABSTRACT

Novel techniques and instrumentation are disclosed for repositioning an intraocular lens (IOL) that becomes dislocated, such as following cataract surgery. In some methods, a trocar or sheath is placed through the eye wall near the ciliary body until a distal tip of the instrument is near the intraocular target to be moved. A clip or other engagement structure at the end of the instrument is manipulated to engage a portion of the IOL, such as a haptic, using various disclosed engagement mechanisms. The instrument is then manipulated to reposition the target to a desired position. In some cases, sutures are attached to the target or to the clip and then secured on the external surface of the eye to secure the target in the desired position.

### CROSS-REFERENCE TO RELATED APPLICATIONS

This application claims the benefit of U.S. Provisional Patent Application No. 61/774,792, filed on Mar. 8, 2013, and entitled “INTRAOCULAR LENS FIXATION CORRECTION METHOD AND DEVICE,” which is incorporated by reference herein.

### FIELD

The present disclosure relates to eye surgery, particularly to methods and devices for correcting dislocation (such as subluxation) of an intraocular lens, such as following cataract surgery.

### BACKGROUND

Cataract surgery is a very common surgery performed in the United States. During cataract surgery the native lens is removed from the patient's eye and replaced with an artificial intraocular lens (IOL) that is made of a plastic or hydrogel material. An IOL generally has two components: a central optic (the lens) and arms (called haptics) that hold the lens centered in the capsular bag or the ciliary sulcus of the eye. Although cataract surgery has a very high success rate, certain complications may occur. One such complication is post-surgical dislocation or subluxation of the lens in which the IOL becomes decentered away from the visual axis or into the posterior segment. Subluxated IOLs can produce such extreme decentration that the IOL optic covers only a small portion of the pupillary space. Luxation involves total dislocation of the IOL into the posterior segment.

Decentration of an IOL may occur because of the original surgical placement of the lens, or it may develop in the postoperative period due to external forces (such as trauma or eye rubbing) or internal forces (such as scarring, zonular weakness, capsular contraction, or size disparity). The majority of patients with posterior IOL dislocations are treated surgically using either a limbal or a pars plana approach. If the IOL is still supported to some degree by the capsular remnants, an anterior-segment (limbal) approach may be considered. When the patient is supine on the operating table the IOL frequently moves posteriorly, which renders a limbal approach more difficult. Pars plana vitrectomy (PPV) techniques are therefore generally used to correct the subluxation. Many subluxed lenses are left untreated until they are severely dislocated due to the difficulty of the current surgical correction procedures. Deferring surgery leaves the patient with suboptimal vision due to induced astigmatism and higher order aberrations from the decentered IOL, and can lead to more complications if the lens dislocates posteriorly into the vitreous cavity.

Current techniques for correcting IOL dislocation are laborious and difficult. These procedures are often performed by anterior segment specialists or retina surgeons because the procedures require specialized skills that are more generally employed and practiced by sub-specialist surgeons.

### SUMMARY

Several exemplary devices and methods are described herein for repositioning and/or securing an intraocular target, such as an IOL, in the eye.

In some disclosed methods, an introduction instrument is inserted through a wall of the eye into or near the ciliary sulcus to access the IOL. The introduction instrument is then used to engage a haptic of the IOL and reposition the IOL within the eye by moving the engaged haptic with the introduction instrument. The introduction instrument is used to position a rigid clipping mechanism, or clip, at its distal end over a haptic of the IOL. The clip includes a discontinuity (for example a separation between first and second arm portions) that opens to receive an intermediate portion of the haptic (as opposed to passing a continuous loop over the end of the haptic). The discontinuity can then be closed such that the clip forms a continuous rigid engagement around the haptic to allow the user to control and reposition the haptic. A similar method can also be used at an opposing side of the eye to engage a second haptic of the IOL to further reposition the IOL.

Engaging the haptic can comprise securely clamping or grasping the haptic with the instrument, and/or positioning a suture around the haptic, such as for moving the haptic or tethering the haptic to the eye wall. Repositioning the haptics often entails moving the haptic from a dislodged position to the properly seated position in the ciliary sulcus, and can also entail securing the haptic using sutures.

In some methods, engaging a haptic of the IOL comprises opening an engagement portion of the clip within the eye, moving the open engagement portion over the haptic, and closing the engagement portion to capture the haptic of the IOL. In such methods, when the haptic is positioned within the clip, the method can also include using the instrument to advance a suture over the haptic and the engagement portion, retaining the suture to a distal end portion of the clip, opening the engagement portion of the clip, and retracting the clip such that the haptic exits the engagement portion and the suture becomes looped around the haptic. The method can further comprise closing the clip with the suture looped around the haptic, pulling the closed clip and a portion of the suture out through the eye wall, and securing the suture to the eye wall to tether the haptic to the eye wall.

In some methods, advancing the suture over the haptic and the engagement portion and retaining the suture to the distal end portion of the clip can include advancing a needle of the introduction instrument through a resiliently deformable plug in the distal end portion of the clip such that the needle carries the suture through the plug, and retracting the needle from the plug such that the suture remains retained by the plug.

Opening the engagement portion of the clip can include retracting an introduction sheath of the introduction instrument to uncover a pre-bent portion of an arm extending through the sheath. The pre-bent portion can be held in a straightened configuration when covered by the sheath, and the pre-bent portion can be allowed to resiliently return to a bent configuration when uncovered by the sheath.

A related device for repositioning IOL in the eye (such as with the disclosed methods) includes a frame adapted to be held by a surgeon, an elongated insertion sheath extending from the frame and adapted to be inserted through an eye wall into the eye, an actuator coupled to the frame and adapted to control longitudinal movement of the insertion sheath relative to the frame, and a haptic engagement portion, or clip, that extends through the insertion sheath and is adapted to open and close to grasp a haptic of the IOL. When the device is inserted into the eye adjacent a target haptic, actuation of the actuator causes the insertion sheath to retract relative to the engagement portion such that the engagement portion resiliently opens to receive the haptic, and releasing the actuator causes the insertion sheath the advance relative to the engagement portion such that the engagement portion closes and grasps the haptic.

The engagement portion can include a first arm and a second arm, the second arm having a pre-bent portion that causes an end portion of the second arm to resiliently move apart from the first arm when the pre-bent portion is uncovered by the insertion sheath. The end portion of the second arm can comprise a pointed distal end adapted to be inserted smoothly through a previously created wound or opening in the eye wall, such as formed with an MVR blade. The end portion of the second arm can further comprise a plug that is positioned in longitudinal alignment with an end of the first arm. The first arm can include a needle sheath and a needle that is longitudinally slidable within the needle sheath upon actuation of a needle controller (such as a plunger) coupled to the frame outside of the eye. The needle is preloaded with a suture passing through the needle and a free end of the suture can extend out of the distal end of the needle. Actuation of the needle causes the needle to advance from the needle sheath and puncture through the plug, carrying the preloaded suture from the needle sheath through the plug. The plug is adapted to retain the suture after the needle is retracted back out of the plug and into the needle sheath. With the suture thus retained by the plug, the engagement portion can be opened to release the haptic, which causes the suture to become looped around the haptic so that the suture ends can be drawn out of the eye and secured to the eye wall to retain the haptic.

In some embodiments, the actuator for controlling longitudinal movement of the insertion sheath comprises two flexible finger pads positioned on opposite sides of the frame, a collar mounted around the frame, and links coupling the finger pads to the collar. Squeezing the finger pads toward the frame causes the collar to move longitudinally away from the eye wall relative to the frame and thereby retract the insertion sheath relative to the frame.

In some disclosed methods, an introduction instrument such as a trocar is introduced through a wall of the eye adjacent or into the ciliary sulcus. A clip is advanced through the introduction instrument to engage a portion of the IOL and move and/or secure the IOL to a desired position within the eye. In some embodiments the clip includes jaws having at least one movable arm that moves between an open clip position and a relatively closed clip position, and the method further comprises advancing the clip through the introduction instrument, deploying the clip from a distal end of the introduction instrument, moving the clip to the open clip position with the arms around a portion of the IOL, closing the arms to a relatively closed position to engage the portion of the IOL, and moving the clip to reposition the IOL. A suture is attached to the clip and extends through the introduction instrument and external to the eye. The suture can be tied externally to the eye to retain the clip against a wall of the eye.

In some disclosed methods, a clip is advanced through an introduction instrument in a relatively closed position. After deployment of the clip from the distal end of the introduction instrument, the arms are moved to a relatively open position. For example, the arms may be jaw members and the open position may be defined by opposing jaw members moving away from one another to present a discontinuity (space) at the end of the clip between the open arms through which the target is moved between the arms of the clip. In other embodiments, one of the arms includes a deformable portion that bends to open as the arm engages and is pressed against the target until the deformable portion of the arm opens to provide a discontinuity in the perimeter defined by the arms to allow the target to move within the perimeter. Once the target is within the perimeter, the pressure on the arm is relieved and the arm closes to again define a closed perimeter around the target for retaining it securely therewithin.

The suture may be tied to a bolster placed on the external surface of the eye over a site where the introduction instrument was introduced into the eye.

Several exemplary clip embodiments are disclosed. For example, the clip may be made of a crimpable metallic or plastic material that is compressed or deformed to conform to the surface or the three-dimensional topography of the target, for example to secure the clip to the IOL. In other embodiments, the clip is made of a resilient material having a shape controllable by the application of pressure to the clip, wherein the application of pressure to the clip opens and/or closes at least one arm by providing a discontinuity in the arm that opens the perimeter of the clip and allows a portion of the IOL to move into the perimeter of the clip; application of pressure to the clip is subsequently changed to close the discontinuity in the arm. In other embodiments, opposing rigid jaw members are movable between an open position in which the jaw members are moved apart (for example away from one another around a hinge to define a V-shaped opening) and a closed position. The open jaw members provide a discontinuous perimeter into which the target is moved. The jaw members are then closable to engage the target (such as an IOL haptic) such that the jaws are secured to the portion of the IOL.

In some embodiments, the device includes opposing first and second rigid jaw members that are positionable around a portion of the IOL. The first jaw member contains a selectively closeable port, and the second jaw member carriers a memory alloy member advanceable from the introduction instrument to move around a haptic of the IOL. The memory alloy member is capable of carrying a suture around the haptic and through the port such that the port may be selectively closed on the suture to retain the suture in engagement with the first jaw member. The first jaw member is configured to carry a suture loop through which the suture retained in the closed port is pulled to tie a surgical knot.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is an elevational view of an intraocular lens having haptics to hold the lens in the ciliary sulcus or capsular bag.

FIG. 2 is a schematic cross-sectional view of a human eye following cataract surgery. An IOL is shown in the posterior chamber of the eye with the haptics of the lens positioned and retained in a peripheral groove known as the ciliary sulcus between the iris and the ciliary body.

FIG. 3 is a schematic front view of a post-operative eye showing subluxation (dislocation) of an IOL. The illustrated dislocation is a nasal decentration, the decentration may be in any direction such as temporal, superior or inferior. In some examples posterior dislocation of the lens also occurs but the presently disclosed methods and devices are particularly useful to reposition the lens and fix it in a desired position prior to signficiant posterior displacement of the IOL into the vitreous that would require more extensive surgery to reposition the IOL. The embodiments may also be used in cases of posterior displacement, such as once a retina surgeon lifts the lens forward from the vitreous into the ciliary sulcus.

FIG. 4A is a schematic cross-section of a post-operative eye schematically illustrating a disclosed procedure to reposition the dislocated IOL by advancing a repositioning instrument to the IOL anterior to the vitreous, fastening the instrument to the IOL, and moving the IOL to a centered position within the eye.

FIGS. 4B, 4C, 4D and 4E schemnatically illustrate use of an alternative version of the instrument to reposition a dislocated IOL. FIG. 4B illustrates attachment to a first haptic and FIG. 4C illustrates suturing the first haptic to the wall of the eye. FIG. 4D illustrates attachment to a second haptic, and FIG. 4E illustrates suturing the second haptic to the wall of the eye. These figures are not drawn to scale.

FIGS. 5-19 depict multiple alternative embodiments of fasteners for securing and moving an IOL. These drawings are similarly not drawn to scale.

FIG. 20 illustrates an alternative use of the devices disclosed herein to repair a traumatically retracted iris (iridodialysis).

FIGS. 21-28 show additional embodiments of repositioning devices.

FIG. 29 illustrates bolsters on the surface of the eye to support sutures that secure the repositioned IOL.

FIG. 30 is a schematic cross-section of a human eye illustrating an endoscope introduced into the ciliary sulcus to view the repositioning instrument in an opposite portion of the sulcus.

FIG. 31 is a schematic cross-section similar to FIG. 30 but showing the endoscope attached directly to the repositioning instrument.

FIGS. 32A-D illustrate an embodiment of the positioning instrument in which a memory alloy needle moves a suture around the haptic of an IOL to tie a surgical knot.

FIGS. 33A and 33B show another embodiment of the repositioning instrument that uses a memory alloy needle.

FIG. 34A is perspective view of another exemplary repositioning device.

FIG. 34B shows a distal end portion of the device of FIG. 34A.

FIG. 35 shows the distal end portion of FIG. 34B entering an eye through the scleral wall toward an IOL.

FIG. 36 shows the distal end portion opening to receive a haptic.

FIG. 37 shows the distal end portion closing to grasp the haptic.

FIG. 38 shows a needle extending with a suture distally through a plug in the distal end portion.

FIG. 39 shows the suture remaining extending through the plug after the needle has retracted.

FIG. 40 shows the distal end portion opening again to release the haptic with the suture extending around the haptic.

FIG. 41 shows the distal end portion closing again to retract the instrument out of eye with the suture around the haptic, such that the suture can be tied or secured outside of the eye to retain the haptic in a desired position.

FIG. 42 is a perspective view of another exemplary repositioning device.

FIGS. 43 and 44 are orthogonal side views of the device of FIG. 42.

FIG. 45 is a cross-sectional view of the device taken along section line E-E of FIG. 43.

FIG. 46 is an enlarged view of section G of FIG. 43.

FIG. 47 is an enlarged view of section H of FIG. 44.

### DETAILED DESCRIPTION

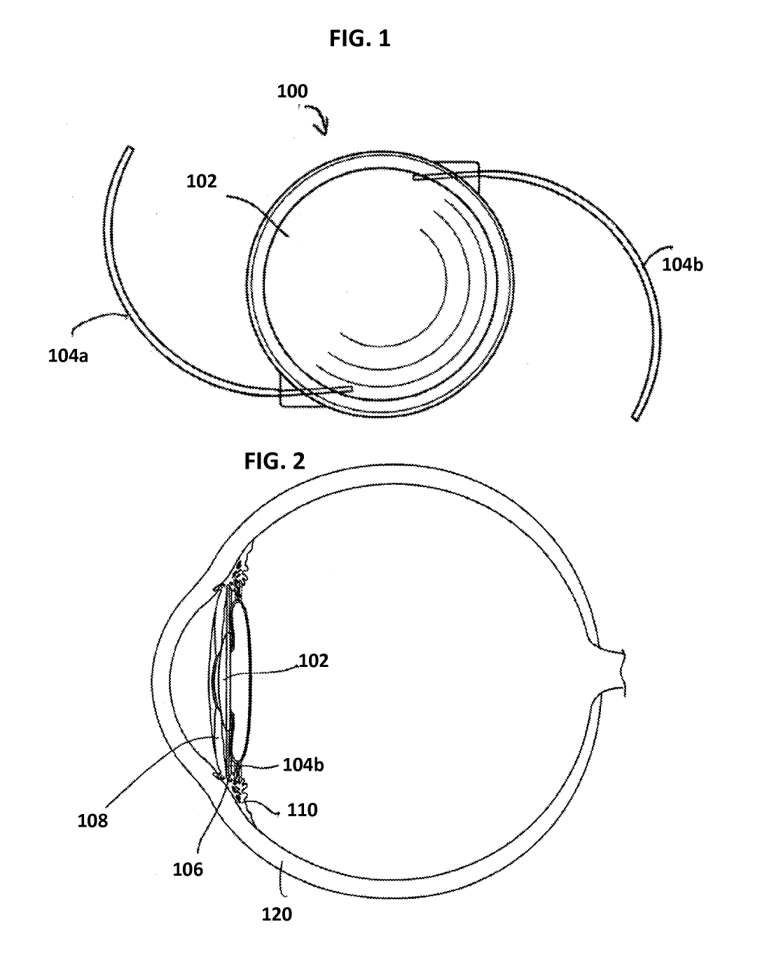

During cataract surgery the native lens is removed from the patient's eye and replaced with a replacement intraocular lens (IOL). An exemplary IOL 100 is shown in FIG. 1 having a central optic 102 (the lens) and two haptics 104a, 104b that project from the lens to hold it in the ciliary sulcus of the eye with an optical axis of the optic substantially aligned with the visual axis of the eye. FIG. 2 illustrates the IOL 100 positioned in the ciliary sulcus 106, which is a peripheral groove between the iris 108 and the ciliary body 110.

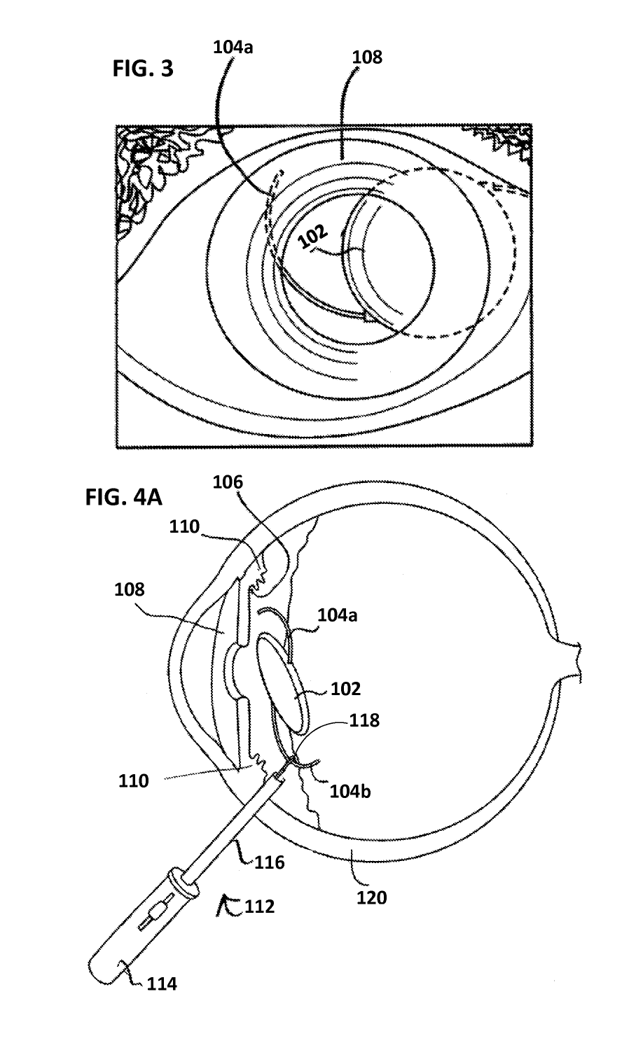

The IOL 100 is sometimes post-operatively dislodged from ciliary sulcus 106, which causes the lens to move off the visual axis as shown in FIG. 3, and the patient loses visual acuity. FIG. 3 shows how the lens looks if it becomes nasally decentered (displaced within the eye toward the nose of the patient). The curved edge of the lens is seen forming an arc along the central visual axis of the eye that interferes with vision. It then becomes necessary to reposition IOL 100 in the ciliary sulcus 106 with the optical axis of IOL 100 properly aligned with the visual axis.

The disclosed technology can also be used to correct so-called “in-the-bag” IOL dislocations, which can include situations where the IOL was originally placed in the capsular bag during surgery, but due to zonular weakness of the capsular bag and/or other causes, the IOL and the capsular bag dislocate together over time. This can occur due to pseudoexfoliation, for example.

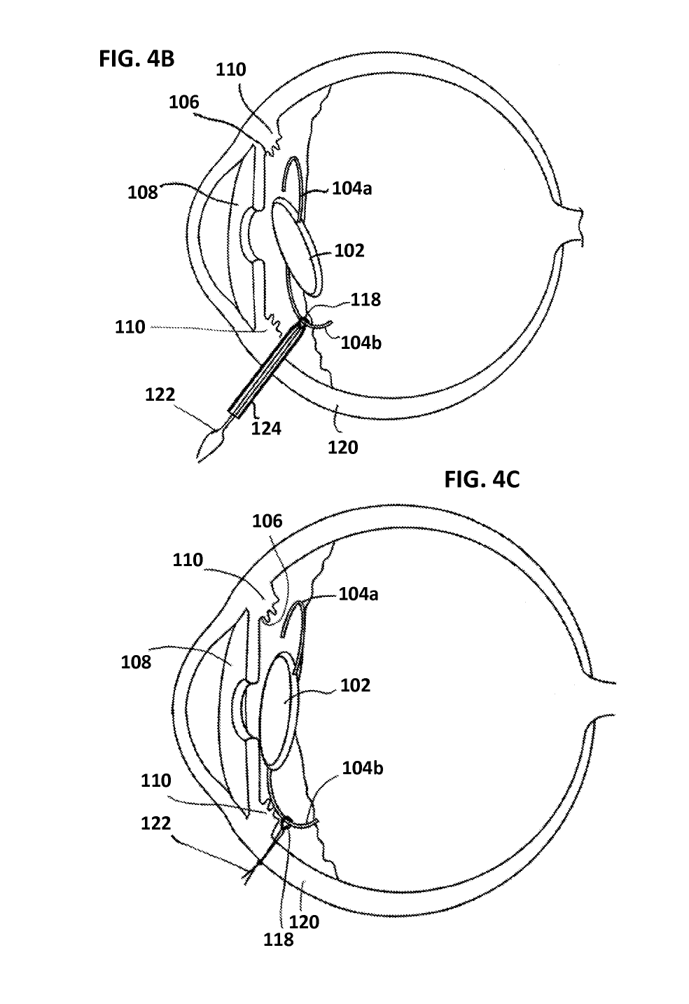

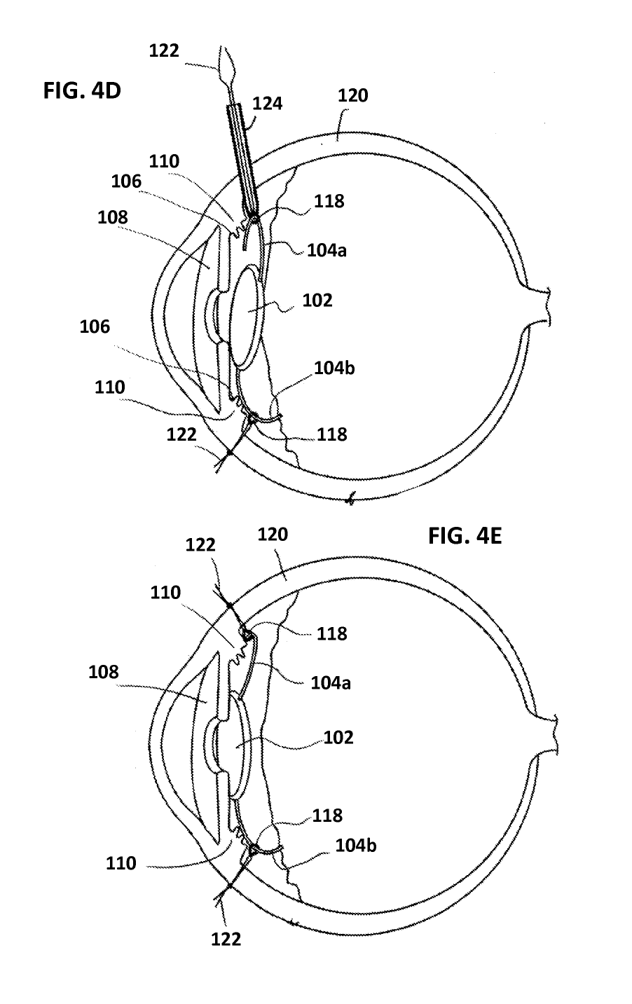

Various repositioning IOL instruments are disclosed herein. An exemplary repositioning instrument 112 is shown in FIG. 4A for repositioning the lens by inserting instrument 112 directly into the eye to grab one of the haptics 104a or 104b for repositioning and fixing the lens 102 in a desired position. The instrument and method can take a variety of forms, such of which are disclosed and described herein. Exemplary detailed surgical procedures for using the instrument are as disclosed, such as those described in the numbered examples below.

FIG. 4A conceptually depicts one embodiment of an elongated surgical instrument 112 that has an enlarged proximal end 114 to be grasped by a surgeon and a smaller diameter portion that functions as a trocar 116 (tube) for introduction of an IOL repositioning device into the eye. A fastener 118 (such as a loop or clip) is schematically depicted at the distal end of instrument 112. The trocar 116 is adapted to be inserted through the wall of an eye, or sclera, 120 just exterior to the ciliary sulcus. An introducer such as a rod is moved through the trocar to advance the fastener 118 that may be radially compressed (in the direction of the radius of the cylindrical trocar) as the fastener moves through the trocar. The fastener 118 may be radially decompressed as it is deployed from the distal end of the trocar 116 to grab the haptic 104b of the decentered IOL. The trocar and clip can then be retracted to pull the haptic back toward the wall of the eye, for example into the ciliary sulcus 106 where the haptic can be sutured to or within the external wall of the eye for retention of the IOL in its proper position.

The fastener at the distal end of the instrument can take a variety of forms, some of which are radially compressed by the trocar and others which are not compressed.

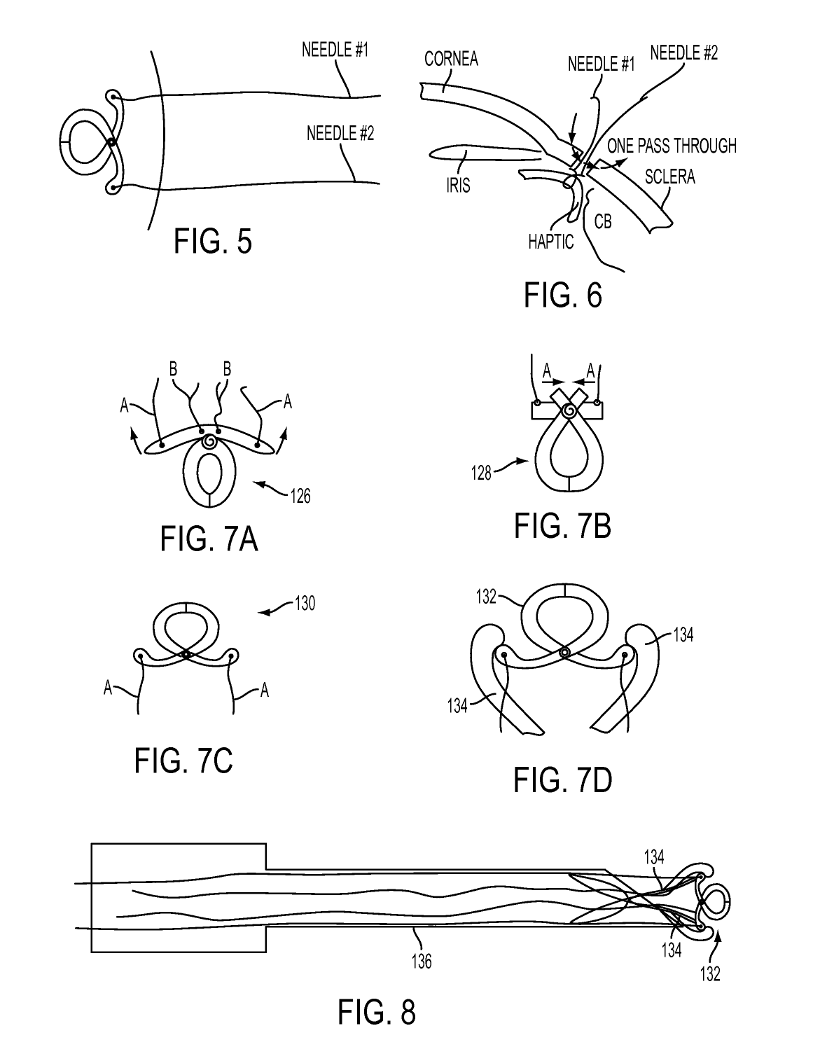

One embodiment of this technology is schematically shown in FIGS. 4B-E, in which the instrument includes a hollow cylindrical trocar 124 introduced through the wall 120 of the eye. The clip 118 comprises two leading arms of a jaw-like member and two trailing legs that each have an eyelet. A suture 122 is attached to each eyelet and the sutures extend substantially parallel to one another through the trocar 124 and out of the proximal end of the trocar outside the eye (FIG. 4B). The enlarged view of the clip in FIG. 5 illustrates an embodiment in which the clip includes hinged arms that are biased to the closed position. The clip is inserted into the hollow cylinder of the instrument and advanced through the instrument by a pusher (not shown) within the trocar. As the clip emerges from the distal tip of the instrument the clip can be opened by exerting tension on the sutures while continuing to exert pressure at the fulcrum or pivot point of the clip with the pusher. The distal tip of the trocar is positioned adjacent a haptic of the IOL prior to pushing the clip out of the instrument such that the open arms of the emerging clip surround its target. Discontinuation of the tension on the sutures permits the arms of the clip to close around the target and secure the clip to the target, as shown in FIG. 6.

After the clip 118 engages the IOL haptic 104b, the instrument is partially or completely withdrawn toward the wall 120 of the eye. The trocar 124 may be removed from the eye with the free ends of the sutures 122 retained external to the eye. Tension is exerted on the sutures 122 until the clip 118 engages the internal wall of the eye at a desired location immediately internal to the wound through which the trocar 124 was introduced. The sutures 122 are then tied to or on the exterior wall of the eye to retain the clip against the interior wall of the eye with the haptic secured to the eye by the sutured clip, as shown in FIG. 4C. A spatulated needle can be provided at the free end of one of the sutures (see FIGS. 5-6) to facilitate grasping the suture and tying the knot. A silicone bolster (see FIG. 29) can also be positioned between the suture and the underlying surface of the eye to minimize pressure of the suture against the underlying tissue and facilitate tying the knot since both ends of the suture extend through the same wound in the wall of the eye.

The opposite haptic 104a can also be repositioned in the same manner, as shown in FIGS. 4D and 4E, to retain the optic 102 in the desired position.

Additional embodiments of clips are shown in FIGS. 7A-7D. All of them are suitable for introduction through a trocar-like access port (e.g., 23-25 gauge) such as the type used in retinal-vitreal procedures. The clips 126, 128, 130, 132 include two generally C-shaped opposing mirror image arms joined at a pivot point, and the arms are generally symmetric with respect to a plane of symmetry through the pivot point. These clips also include two legs that extend from the pivot point in a direction generally transverse to the plane of symmetry. In the illustrated embodiments, each leg is a continuation of one of the arms. Each leg is attached to a suture (such as sutures made of Gore-Tex® or Prolene). The natural conformational state of the clips is closed as shown in FIGS. 7A-7D, but the clips can be opened by exerting on the arms of the clips a force in the direction shown by the arrows in FIGS. 7A-7D. Once the arms are opened the clips can engage a haptic and then be closed to secure the clip to the haptic by tying either sutures A or B to the wall of the eye.

The clip 132 shown in FIG. 7D can be introduced through a hollow trocar. As shown in FIG. 8, the legs of the clip 132 engage a flexible holder 134 that flares to greater than the diameter of the cylindrical trocar 136. As the holder 134 is retracted into the distal tip of the trocar 136, the flared ends of the flexible holder 134 are compressed which pulls the legs of the clip 132 toward each other to open the clip and provide a discontinuous perimeter so that the arms of the clip can encircle the haptic. The holder 134 can subsequently be advanced out of the distal end of the trocar 136 so that the flexible legs of the holder move apart and allow the clip 132 to close by returning to its biased orientation. FIG. 8 schematically illustrates this clip 132 as it is advanced through the trocar 136 for deployment.

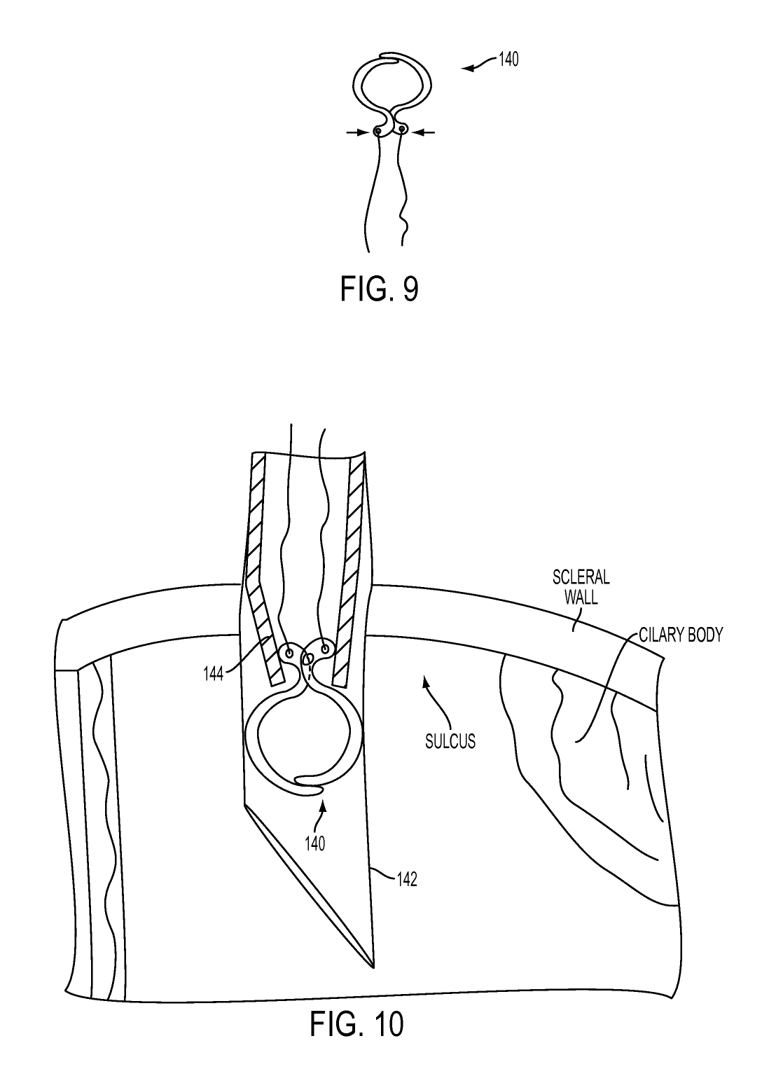

Another exemplary clip 140 shown in FIGS. 9 and 10, which includes overlapping clip arms to provide a smaller proximal end to which the sutures are attached. An advantage of this embodiment is that the distal end of the clip 140 can more easily fit in the ciliary sulcus. Each member of the clip 140 is generally S-shaped with a top curve of the S larger than the bottom curve of the S. The members of the clip 140 are joined at a hinge where the large and small curve of each S-shaped member meet, but with one S-shaped member over the other such that they are in parallel planes. The leading ends of the arms also overlap as shown in FIG. 9.

Use of the clip 140 is illustrated in FIG. 10. A trocar 142 is inserted through the wall of the eye and the clip 140 is advanced through the trocar by a holder 144 that can also squeeze the hinged legs of the clip to open the arms of the clip to allow them to encircle the haptic.

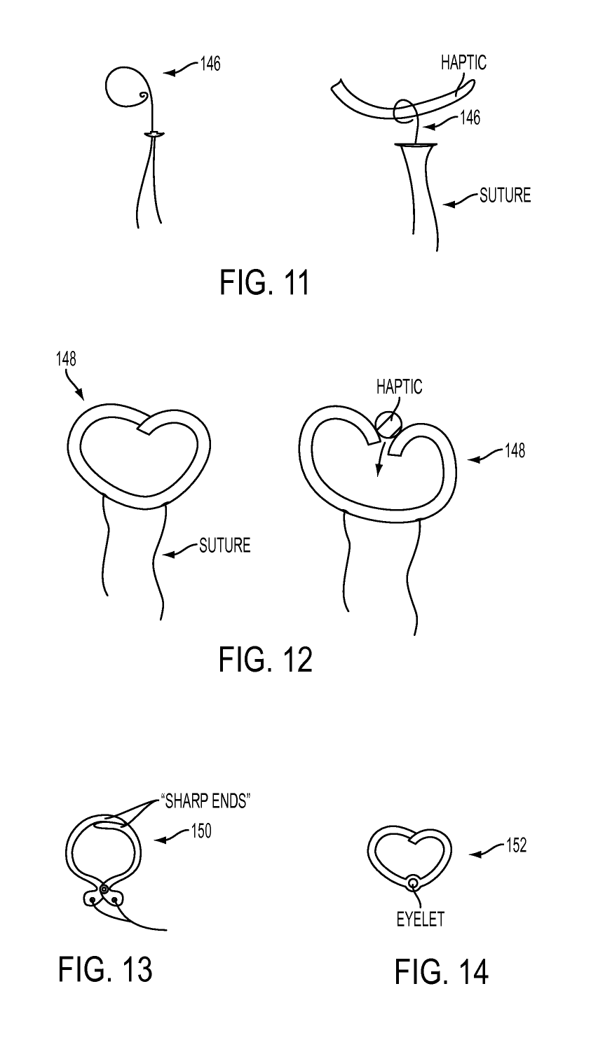

FIG. 11 shows another exemplary clip 146 made of a shape-memory material on the tip of a rod that is capable of being deformed to a generally linear configuration and moved longitudinally within the trocar. The clip 146 curves when it is not held within the hollow core of the trocar, and sutures are attached to the memory shape clip. Once the instrument is introduced into the eye, the clip 146 is advanced out of the tip of the instrument (for example with a pusher within the trocar) to loop around the haptic and grab it to reposition the IOL. Once the IOL is repositioned, the trocar is withdrawn from the eye and the sutures attached to the clip 146 are tied to the eye.

FIG. 12 illustrates a “pretzel clip” embodiment 148 in which the plastic overrlapping arms of the clip can be inwardly deformed. The deformable arms of the clip 148 can be pushed against the haptic of a dislocated IOL to inwardly deform the arms until the haptic slides between the deformed arms and is engaged within the arms of the clip.

FIG. 13 illustrates a clip embodiment 150 having overlapping arms with sharpened arm tips that can puncture residual capsule material as needed. A proximal end of the clip 150 has pivotally engaged legs with eyelets to which suture is attached prior to introduction into the eye.

FIG. 14 shows a clip embodiment 152 similar to the clip 150 of FIG. 13, but the legs of the clip 152 have been eliminated. The clip 152 comprises two arms hinged at their common base, and with an eyelet through the base to which a suture is to be attached.

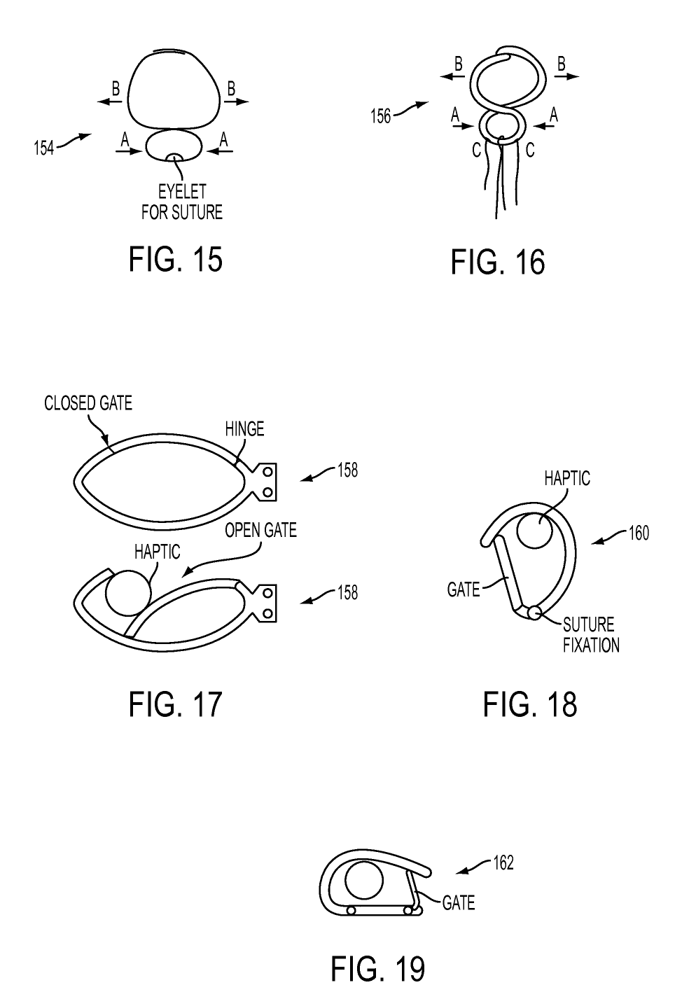

FIG. 15 shows a “figure-8” hingeless clip embodiment 154 comprising resilient overlapping arms and legs that are configured into the shape shown in the drawing. The legs also form an eyelet to which a suture can be attached. Pressure exerted in the direction of A on the legs causes the arms to selectively open the perimeter of the clip 154 in the direction of B to encircle and grasp a haptic. The pressure A can, for example, by exerted by a forceps-like device, such device 144 shown in FIG. 10. The force A is then discontinued to close the arms of the clip 154 around the haptic. The instrument is then manipulated to move the clip 154 and its secured haptic/IOL to a desired position where it is sutured or otherwise secured in the new location by a suture through the eyelet.

FIG. 16 illustrates a clip embodiment 156 similar to clip 154 of FIG. 15, but showing suture material looped around the legs at a variety of possible positions.

FIG. 17 illustrates a “gated” clip embodiment 158 having an elongated ovoid shape defined by a first arcuate arm and a second generally mirror image arcuate arm joined at a suture fixation base that defines one or more eyelets (two are illustrated). One of the arms is hinged at or near the base and also contains a discontinuity point within the arm such that pressure against the arm as it abuts a haptic causes inward flexion of the arm to open the perimeter of the clip 158. As illustrated in the lower part of FIG. 17, the inward flexion of the arm allows the haptic to enter the clip 158, which is then slightly retracted (not shown) to move the haptic into a distal end (left side) of the clip. As the haptic moves to the distal end of the clip 158, the haptic clears the hinged arm, which is biased to flex back to its original unflexed position. The haptic is then securely engaged within the closed arms of the clip 158 for repositioning of the IOL.

FIGS. 18 and 19 show two clip embodiments 160 and 162 that are similar to clip 158 of FIG. 17, in which a gated arm opens to selectively allow the haptic to enter the clip, but wherein the arm subsequently closes to secure the haptic for repositioning the IOL.

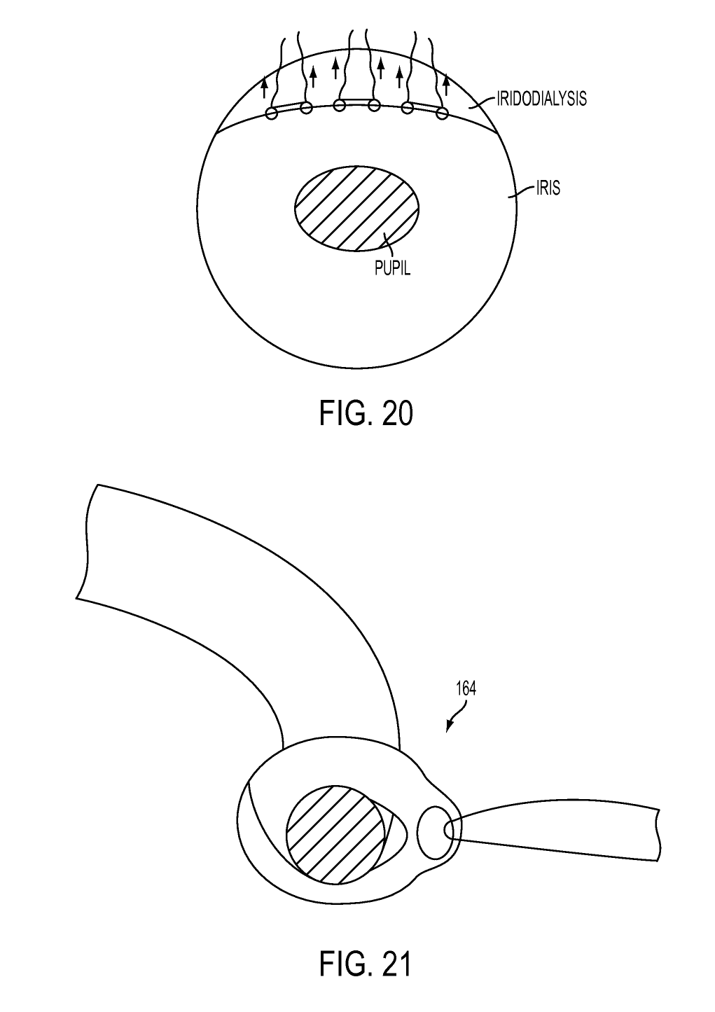

In addition to repositioning dislocated IOLs, the clips or fasteners disclosed herein can be used to mobilize or move other structures within the eye and optionally suture them to the wall of the eye. For example, as shown in FIG. 20, one or more clips can be used to repair an iridodialysis in which there is a localized separation of the iris from the ciliary body that exposes an unsecured edge of the iris. The clips can be attached to the unsecured edge of the iris as schematically illustrated in FIG. 20, and the attached sutures retracted to move the edge of the iris toward the ciliary body or wall of the eye. The sutures can then be tied to secure the iris in that position.

These and other embodiments are further illustrated with reference to the following Examples.

### EXAMPLE 1

### Surgical Procedures

Some exemplary procedures uses a single stab-wound through the sclera into the ciliary sulcus with an instrument similar to instrumentation used in a pars plana retinal surgery. These techniques can be done with or without a trocar, depending on the specific instrumentation selected. These techniques can be performed using either a conjunctival peritomy with a scleral flap or a Hoffman pocket.

Visualization can be achieved using an operating microscope with the patient recumbent. When there is not adequate dilation, an iris hook or hooks (or a Malyugin ring) may be placed to provide direct visualization of the haptic/optic junction. A first hollow instrument (#1), which is used to access the sulcus through the pars plicata, can be a 21-25 gauge hollow shaft with a sharp end to penetrate through the wall of the eye into the sulcus. In a particular example, an Alcon 25 gauge retina trocar is used. The trocar is left in place to provide surgical access to the sulcus. A second instrument (#2) advances one of the clips through the hollow instrument #1 and out the distal end of instrument #1 into the posterior chamber/sulcus region of the eye, where instrument #2 is further advanced so that it and the clip can be visualized through the operating microscope. Once viewed by the surgeon in the intraocular sulcus, instrument #2 is used to open the arms of the clip, for example by applying pressure to the legs of the clip. The open clip is then advanced over and locked around the haptic (or optic edge), and subsequently released by instrument #2 to allow the clip to close, for example by reverting to a biased closed position. Instrument #2 and subsequently #1 are then removed from the eye. The clip remains attached to the IOL, and the attached suture lengths (e.g., 9-0 Gore-Tex® or Prolene) travel out the scleral tract of the original stab incision. Optionally, a bolster (#3; e.g., silicone or plastic) is then threaded onto one or both sutures. The sutures are tied down to the bolster securing the clip/haptic/lens complex to the scleral wall. The same procedure is performed about 180 degrees away at the opposing haptic, ultimately securing and centering the IOL.

In some embodiments, instruments #1 and #2 can be combined into one stab/insertion instrument to advance a clip and secure it to the IOL. This instrument is referred to as instrument #1.5.

### EXAMPLE 2

### Surgical Procedures with Conjunctival Peritomy

In some exemplary procedures, a conjunctival peritomy is performed. A limbus-based ½ thickness scleral flap is created at the pars plicata. The method of Example 1 is then performed. The scleral flaps are closed over the bolster and subsequently the conjunctival peritomy is closed.

### EXAMPLE 3

### Surgical Procedures with Hoffman Pocket

Some procedures are performed with a Hoffman pocket. A Hoffman pocket is a lamellar fornix-based corneo-scleral flap that is started in clear cornea and dissected posteriorly without opening conjunctiva. Two Hoffman pockets are created about 180 degrees from each other at the axis where the haptic is most accessible. A stab incision with instrument #1.5 is made through conjunctiva and through the Hoffman pocket into the ciliary sulcus. The clip is secured to the haptic and instrument #1.5 removed so that the suture emerges through the scleral tract originally made with instrument #1.5. This tract extends through the Hoffman pocket and then out sclera/conjunctiva to the surface of the eye. A hook is used to grab the sutures in the Hoffman pocket and bring them out of the pocket. The bolster is placed on the suture ends and then advanced into the pocket. The sutures are then tied down to the bolster securing the clip/haptic/lens complex to the scleral wall. The same procedure is then performed about 180 degrees away.

Both instruments can also be used to secure intraocular devices such as a modified Cionni capsular tension ring (CTR), Ahmed capsular tension segments, or centering subluxed IOLs in cases where a CTR had been placed (by securing the clip to the CTR/bag complex instead of the haptic to center the IOL). The instrument and clip can also be used for any procedure that requires fixation of an intraocular device to the ciliary sulcus. The clip can be modified to optimize attachment to Cionni tension rings, etc., and their eyelets.

### EXAMPLE 4

### Types of Clips

Clips disclosed herein can secure the haptic near the haptic/optic junction. A suture may be preloaded and attached to the clip or loaded by the surgeon at the time the procedure is performed. In some examples, the clip is configured to either crimp on to the haptic or transiently deform (for example from a biased-closed position) to selectively open and subsequently close for attachment to the IOL.

A clip that crimps can be made, for example, from an inert biocompatible metal such as gold or stainless steel (materials that that have been used with success in the eye). The metal is crimped onto the haptic securely to confirm to the topography of the optic or haptic. Deformable plastic materials can similarly be used in place of a deformable metal. The clip 164 shown in FIG. 21 is an example of a crimping clip.

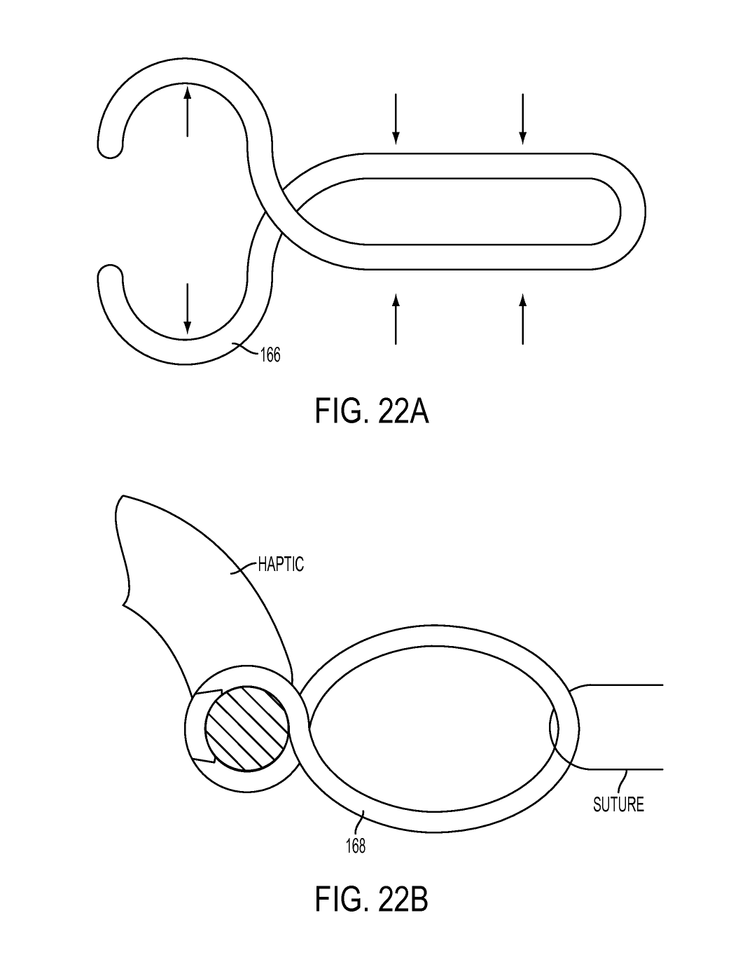

A selectively or transiently deformable clip can be made of a spring-like or resilient material, such as a polypropylene-type material, which in its native or pre-biased state will clamp onto the IOL, or a portion of it such as the haptic. Instrument #2 can deform the trailing (proximal) end of a clip so that a leading (distal) end of the clip can open to enable capture of the haptic. Application of the deforming pressure to the trailing end and opening of the arms of the front end of an exemplary resiliently flexible clip 166 is shown by the arrows in FIG. 22A. The deformation pressure is subsequently released to close the clip 166 so that the arms form a generally circular engagement portion that generally conforms to the shape and dimensions of the haptic that is targeted, thereby securing the haptic to the clip. A similar clip 168 is shown in FIG. 22B secured to a haptic with its arms overlapping. The ends of the arms of the clip 168 that meet and overlap can be tapered to a sharp front edge that is useful to pierce capsular remnants as the arms of the clip close if capsular remnants are surrounding the haptic. A suture is shown looped through the trailing end of the clip 168 for securing the clip to the wall of the eye.

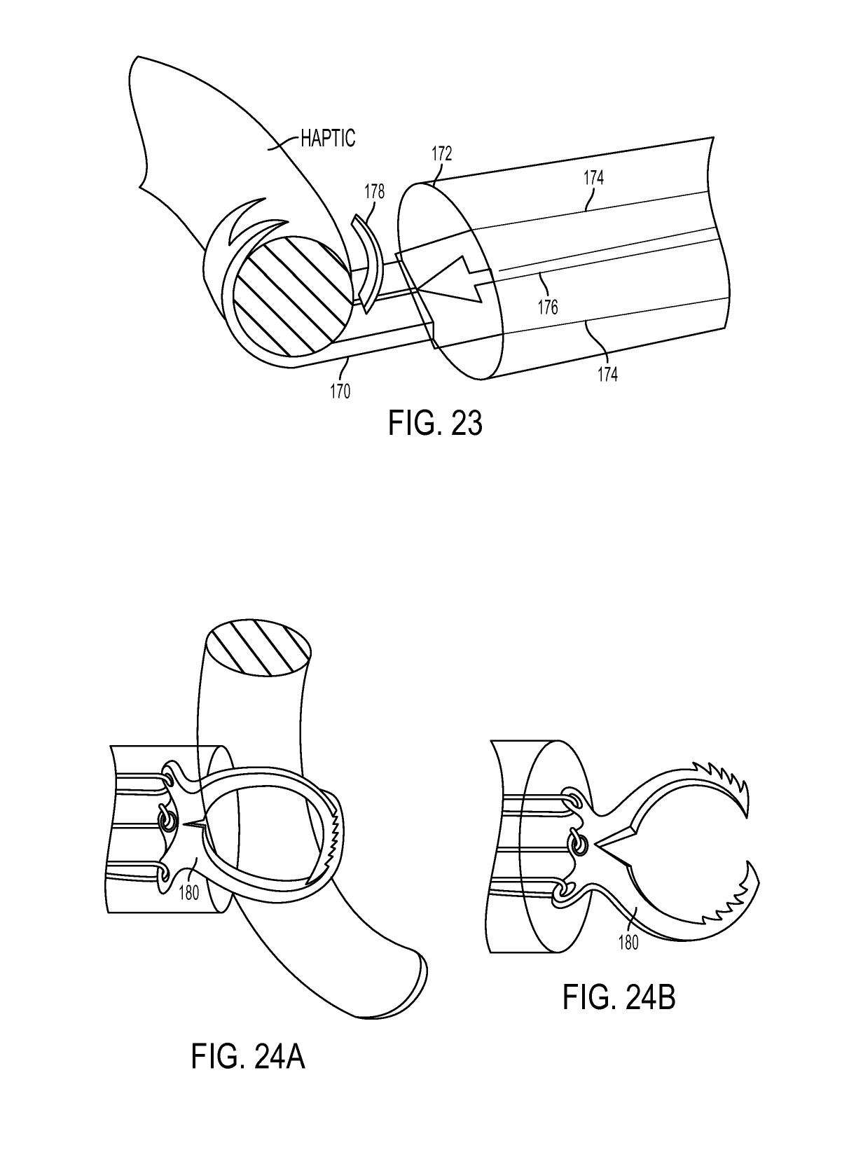

A toothed-clasp clip 170 is shown in FIG. 23 in which the clip comprises a claw-like device 178 advanced through a trocar 172. The inserter instrument within the trocar 172 is used to counter-traction against a suture 174 and subsequently open the device so that the claw 178s close around the haptic, as shown in the direction of the arrow in FIG. 23.

FIGS. 24A and 24B schematically illustrates another clip embodiment 180 having arms that are opened to expose previously overlapping serrated distal arm portions that are useful to puncture through capsular remnants and secure the haptic to the clip.

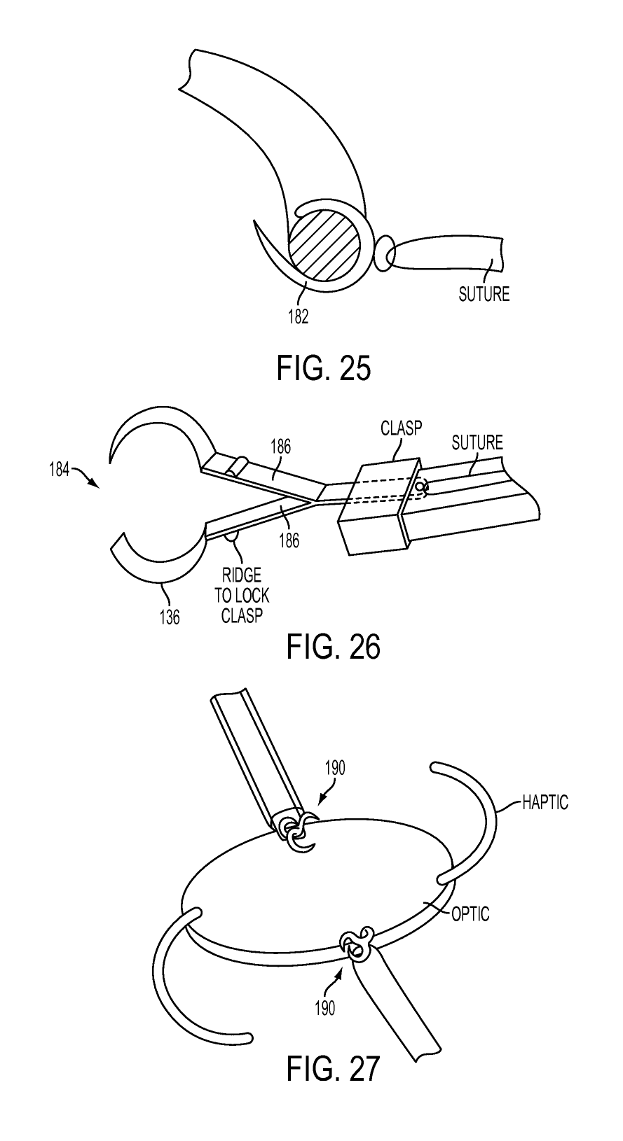

FIG. 25 illustrates a generally J-shaped clip 182 in which the arms can be resiliently deformed to transiently enlarge the opening between the arms. The arms separate until they reach a point of maximum separation when they are apart a distance substantially equal to the diameter of the haptic. As the clip 182 continues to advance over the haptic the arms of the clip are able to move slightly back toward one another to securely clasp the haptic within the arcuate clip.

FIG. 26 schematically illustrates a clasp-locking clip 184 to secure the haptic within arms 186 that are configured and dimensioned to fit tightly around the haptic. The clasp arms 186 of the clip 184 are opened to permit engagement of the haptic, and then the clasp is closed. Opposing surfaces of the clasp arms 186 are configured to snap together tightly to retain the arms in the closed position around the haptic for repositioning the haptic. Sutures are attached to the clasp clip 184 to secure it to the wall of the eye.

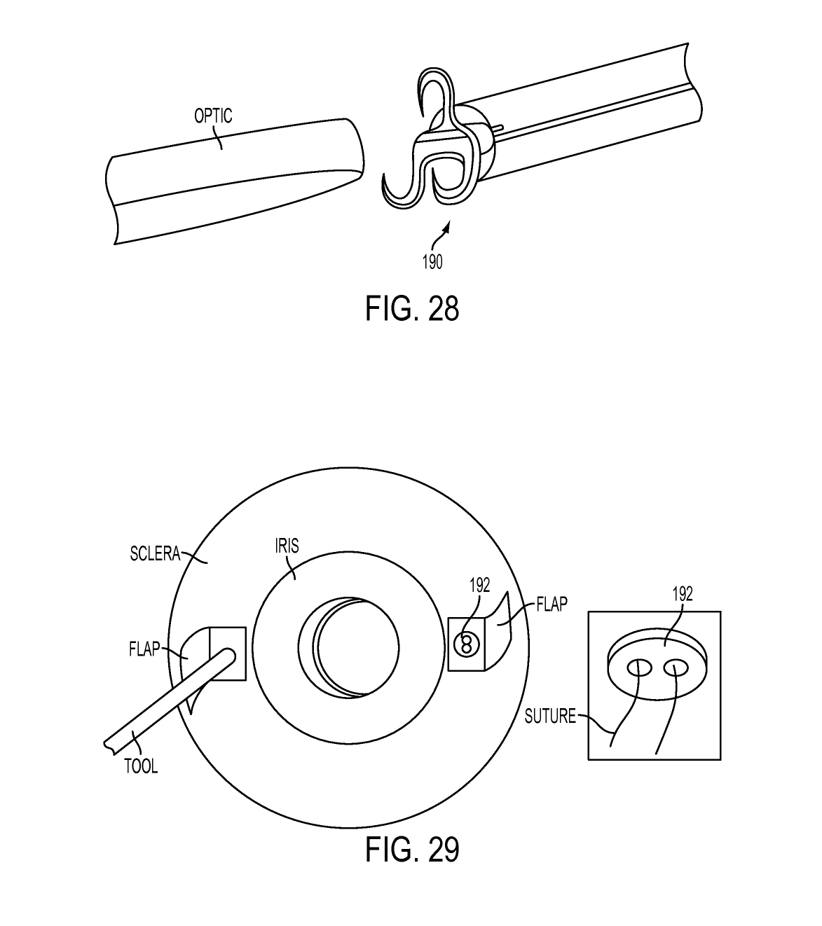

FIGS. 27 and 28 illustrate an alternative clip embodiment 190 that can be used for attachment to the edge of the IOL optic instead of the haptic. A crimping embodiment of the clip 190 is shown which has a plurality of arms with pointed tips that can be selectively closed around an edge of the optic to secure the clip to the IOL. The clip 190 can then be manipulated with the herein disclosed instruments to move the IOL to a desired position and sutures secured to the clip can enable suturing of the clip to the wall of the eye.

### EXAMPLE 4

### Bolsters

FIG. 29 illustrates a bolster 192 that is used in some methods disclosed herein to help secure sutures to the sclera while protecting the underlying tissue. In specific examples, the bolster can be made of silicone or polypropylene, and can be in the shape of a small button or disk with multiple (for example two) holes in it through which the ends of the suture are placed and tied, as illustrated. The bolster 192 is interposed between the finished suture knot and the scleral surface. The bolster 192 is useful to secure the suture to the eye when both ends of the suture emerge through the sclera at the same point.

### EXAMPLE 5

### Endoscopic Visualization

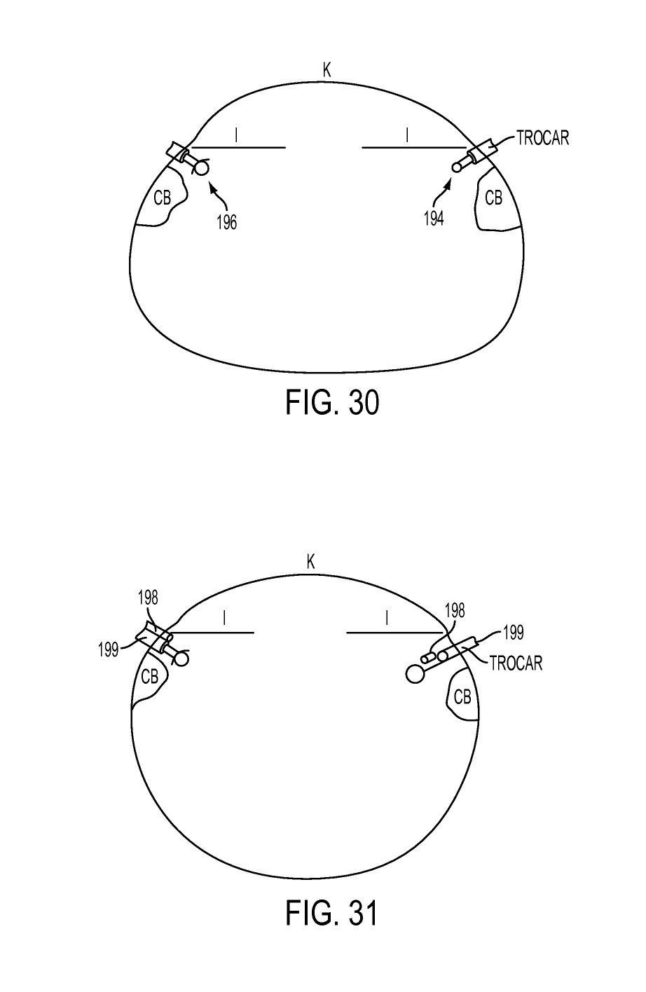

FIG. 30 schematically illustrates in cross-section an endoscope 194 introduced into the ciliary sulcus to view the repositioning instrument 196 in an opposite portion of the sulcus. In the illustrated example, the endoscope 194 is introduced into the ciliary sulcus approximately 180 degrees from the repositioning instrument 196 for visualization of the instrument and clip across the eye.

FIG. 31 illustrates alternative embodiments in which an endoscope 198 is attached directly to the repositioning instrument 199 for visualization of the clip as it emerges from the instrument and is attached to the IOL.

### EXAMPLE 6

### Single Stab Instruments and Procedures

This example describes exemplary single-stab instruments that are capable of passing a suture around a haptic through a single stab incision. Such devices can be adaptable to pass a suture around the haptic of an IOL through a single stab sulcus incision and subsequently suture the haptic of the IOL to the pars plana sclera with a bolster. Such devices can also be used to repair iridodialysis.

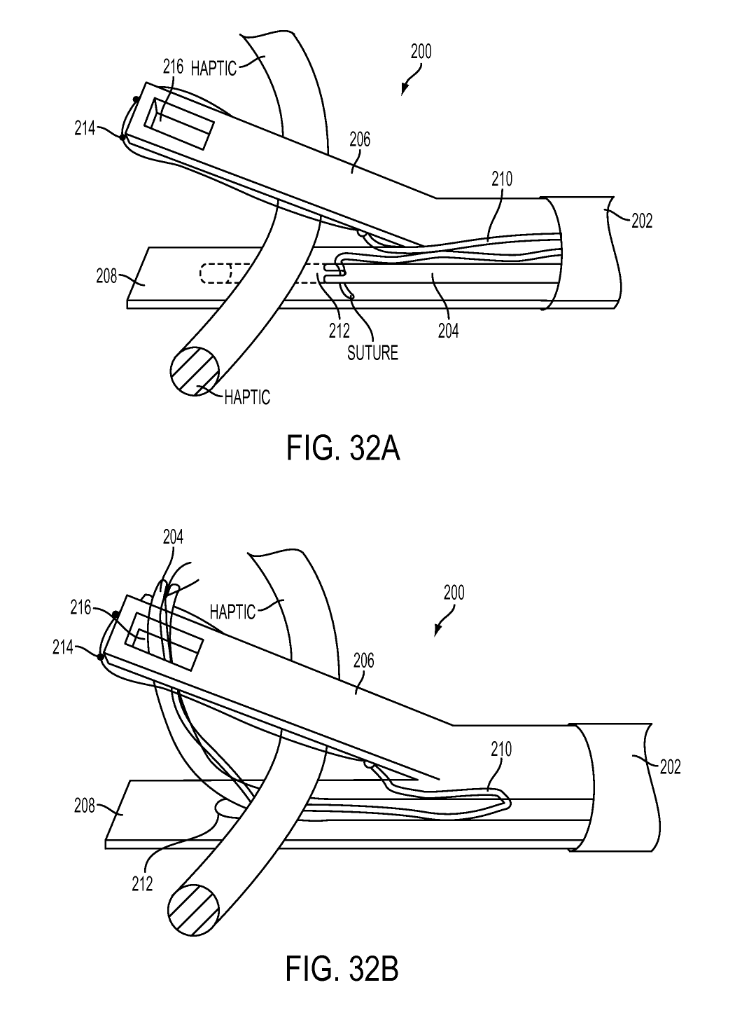

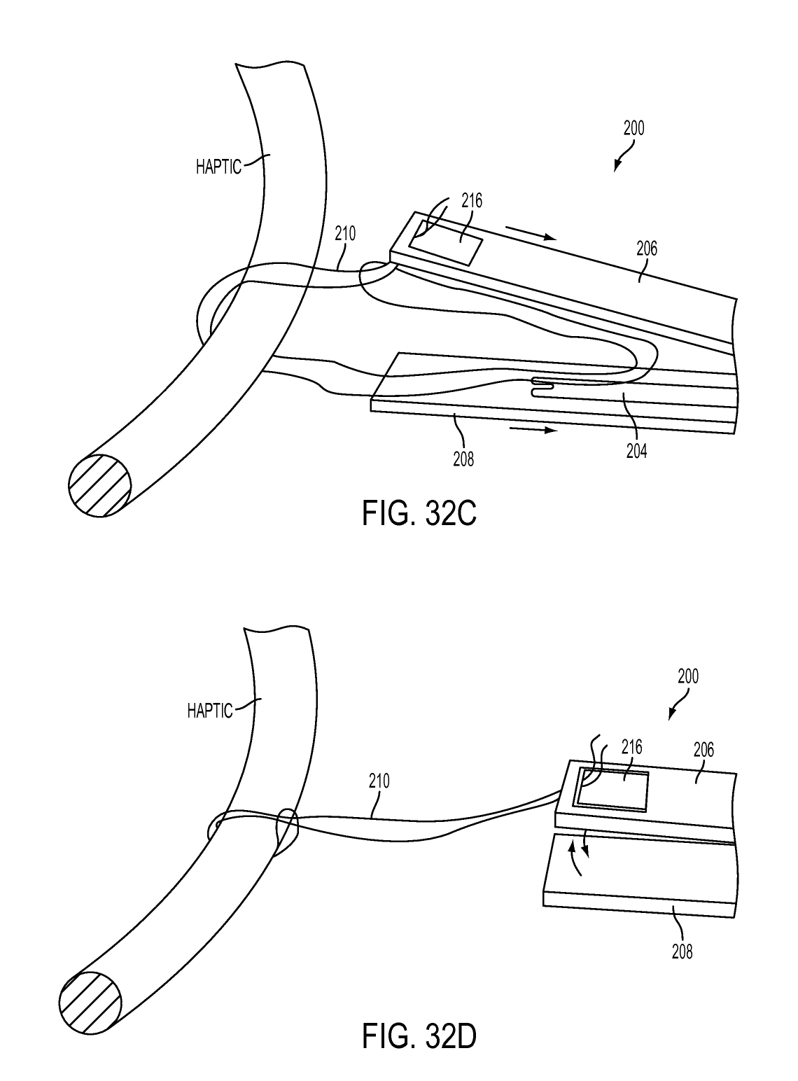

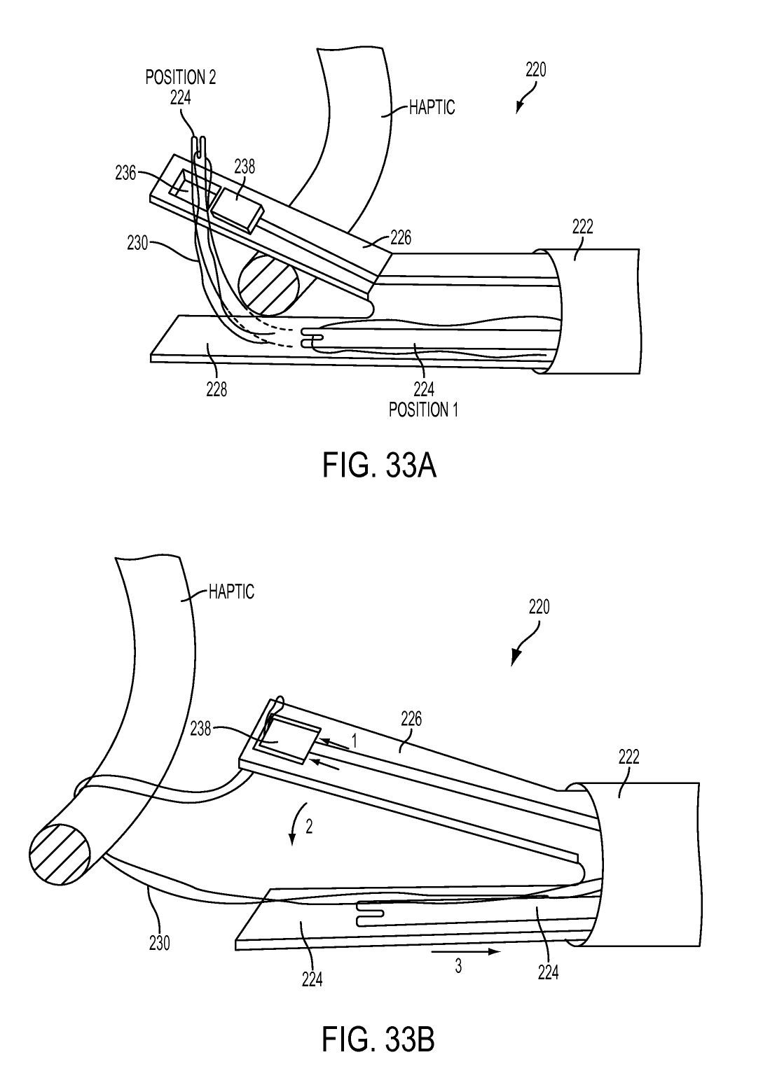

Exemplary single stab devices 200 are shown in FIGS. 32 and 33. A single stab 21-25 gauge needle trocar 202 is placed through the pars plana using a conjunctival peritomy and scleral flap as previously described. Both devices 200 create a cow hitch around the haptic in the eye, using a needle 204 made of shape-memory material, such as a superelastic metal alloy (e.g., nitinol), that passes the suture around the haptic.

As shown in FIG. 32B, the needle's 204 native conformation has an acute bend at its leading (distal) end that is configured to ‘bend’ around and conform to the shape and size of the target haptic. As shown in FIG. 32A, the needle 204 is loaded into a tunnel 212 in the device which retracts the needle and flattens it. The suture 210, such as a 9-0 Gore-Tex® suture, is preloaded into the device 200, which includes opposing jaws 206 and 208 so that a loop of suture is held by jaw 206 in flexible hooks 214 and the trailing ends of the sutures are loaded in the needle 204 in the tunnel 212 in jaw 208. At least one of the jaws of the V-shaped clip 200 is movable relative to the other jaw; in the illustrated example jaw 206 is movable. The clip 200 is closed by moving jaws 206 and 208 towards one another to narrow the angle of the V, and the device is inserted into the eye through the trocar 202. As the device emerges from the trocar and approaches the haptic, jaw 206 is opened and the jaws are placed around the haptic. The jaws are closed and the needle 204 is then advanced as shown in FIG. 32B. As the needle advances, the end portions of the sutures 210 are pulled forward with the needle. Once the needle 204 is advanced out of its tunnel 212, it assumes its native curved conformation, making a steep turn around the haptic, through the suture loop and into the port 216 of jaw 206 (FIG. 32B). The port 216 is then closed as shown in FIG. 32C so that the sutures 210 are trapped in the port as the needle 204 is retracted back into jaw 208. Jaw 206 is then opened from jaw 208 and pulled away from the haptic. The suture ends are now through the suture loop, and as the device 200 is pulled away from the haptic and closed (FIG. 32D), and the suture loop is released from its flexible hooks. The entire device is then retracted out of the eye, pulling the sutures 210 with it and creating a cow hitch on the haptic. The sutures 210 are then tied as previously described, for example with the use of a bolster.

An alternative embodiment 220 is shown in FIG. 33A, which is similar to the device 200 of FIG. 32, but a loop of suture 230 is loaded only in a needle 224 in jaw 228. The suture 230 is advanced around the haptic with the needle 224, then ‘grabbed’ by a trap door mechanism 236 in jaw 226 that is operated by a push/pull member 238 to selectively open or close the trap door (“1” in FIG. 33B). The jaws are then closed (“2” in FIG. 33B), and the device is then pulled from the eye (“3” in FIG. 33B). A cow hitch is then created outside of the eye and the hitch is advanced into the eye until it is tightly secured on the haptic. The sutures are then tied as previously described.

An alternative embodiment of a single stab instrument avoids use of the bolster when both sutures exit the same wound. An MVR (microvitreoretinal) blade can be used to make a stab incision adjacent the exit wound where the two sutures are emerging from the eye (under the scleral flap). A ‘hook-like’ second instrument can then be inserted through the MVR incision, advanced into the sulcus where it is visible, and used to ‘grab’ one of the two sutures, which is then pulled out through the MVR incision. This suture along with the other suture (which remains in the initial exit wound) are then tied and the overlying scleral flap secured.

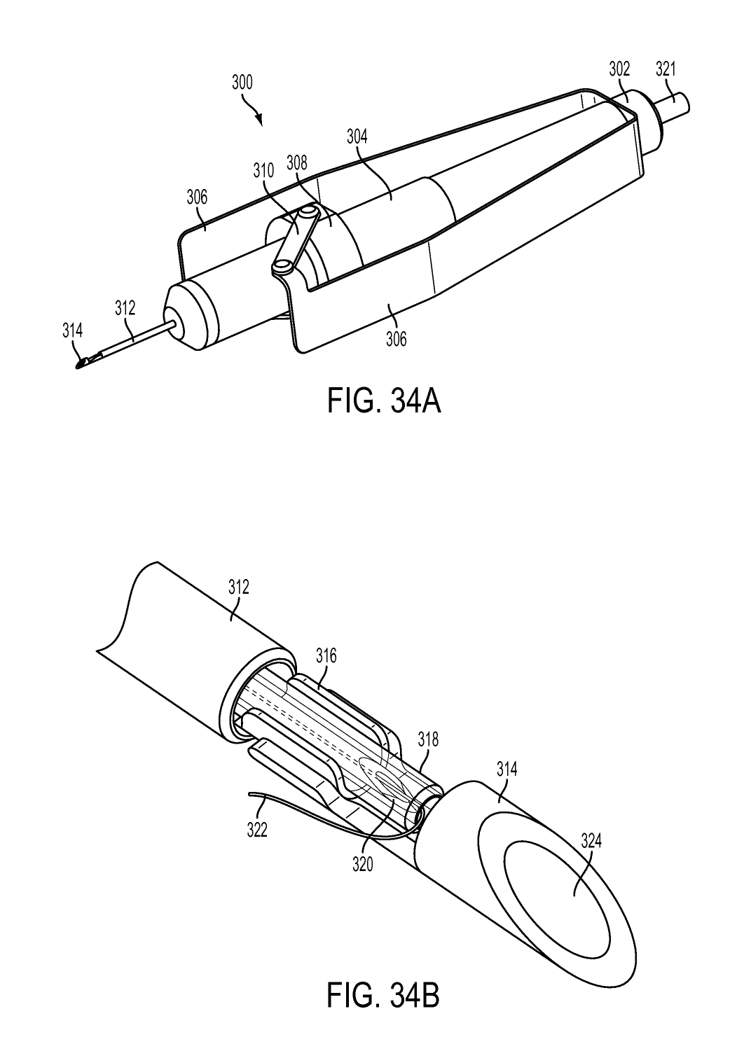

FIGS. 34-41 illustrate another single stab procedure for repositioning and retaining an IOL using an exemplary device 300. As shown in FIG. 34A, the device 300 includes a generally cylindrical or tubular frame 302, an actuator 304 mounted on the frame and adapted to slide longitudinally over the frame, finger pads 306 having a fixed portion mounted on the frame, such as at a rear end, and free ends that can resiliently flex toward the frame and actuator when pinched together by an operator, a collar 308 mounted to the actuator, links 310 that pivotably couple the free ends of the finger pads to the collar, an introduction shaft 312 that extends from the distal end of the frame for insertion into the eye, and a distal body 314 that leads through the eye wall during insertion, capturing a haptic, and placing a suture around the haptic. As shown in FIG. 34B, the distal end portion of the device 300 also includes a carrier 316 extending from within the distal end of the shaft 312, a needle shaft 318 positioned within a trough or bracket at a distal end of the carrier 316, and a hollow needle 320 positioned within a lumen of the needle shaft, with a suture 322 threaded through the needle and extending out of distal ends of the needle and needle shaft. The distal body 314 is fixed at a proximal end to the trough portion of the carrier 316 and has a pointed distal end portion that includes a lumen that is filled with a plug 324. For example, the distal body 314 can be formed from a needle that is larger in diameter than the needle 320. The carrier 316 and distal body 314 form one arm of a rigid clip, while the needle shaft 312 and needle 320 form a second arm of the clip. The clip has a discontinuity between the end of the needle shaft 312 and the distal body 314 that can receive a haptic and can be closed to form a continuous, rigid engagement around the haptic.

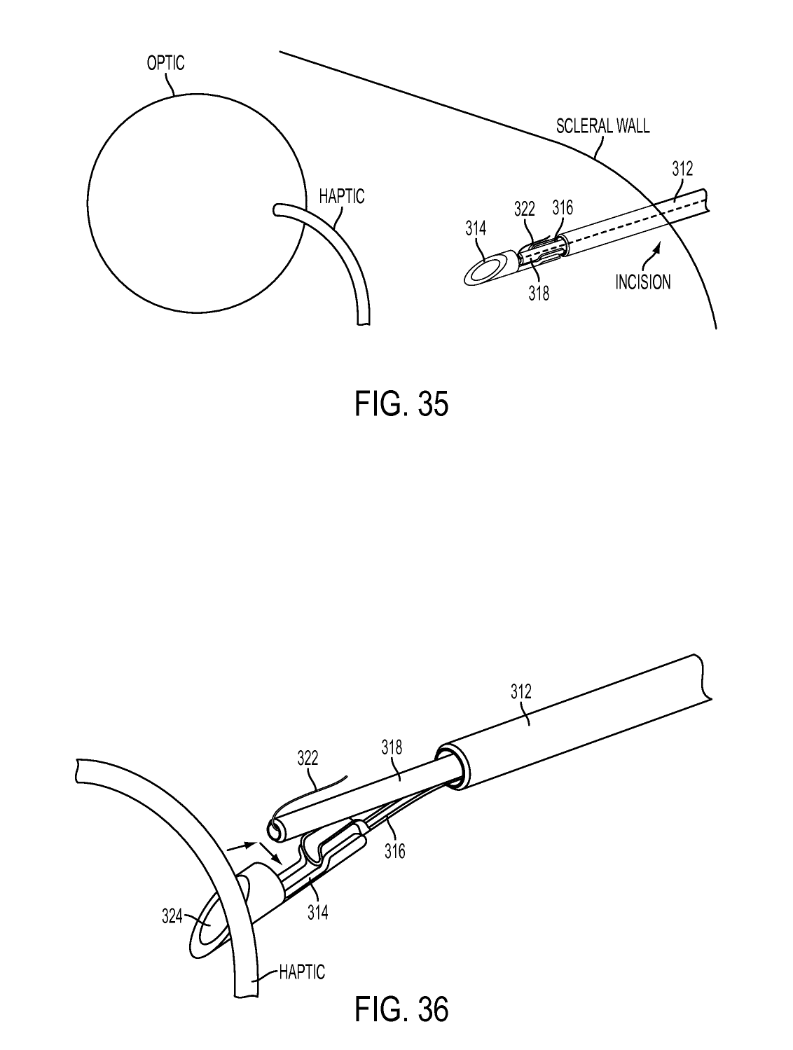

In the configuration shown in FIGS. 34A, 34B, and 35, the device 300 can be inserted through the scleral wall such that the shaft 312 is inserted across the scleral wall and the frame 302, finger portions 306, etc. are outside of the eye, as shown in FIG. 35, either with or without a preliminary incision made in the scleral wall. For example, a preliminary MVR stab incision can be made and the device 300 can pass through the incision. Once inserted into the ciliary sulcus, the operator can pinch the finger pads 306 to cause the actuator 304 to move proximally relative to the frame 302, via the links 310 and collar 308. Proximal motion of the actuator 304 causes the shaft 312 to retract proximally relative to the distal body 314, carrier 316, needle shaft 318, and needle 320, as shown in FIG. 36. The carrier 316 includes a pre-bent portion that is held in a resiliently deformed straightened position within the shaft 312 when the sheath is in the distal position shown in FIG. 35. When the sheath 312 is retracted by pinching the finger pads 306, the pre-bent portion of the carrier is uncovered, allowing the distal body 314 and the end of the carrier distal to the pre-bent portion to resiliently bend away from the needle shaft 318, as shown in FIG. 36. This opens a haptic receiving gap between the distal end of the needle shaft 318 and the distal body 314, as shown in FIG. 36. The device 300 can then be manipulated to cause a haptic to move into a haptic pass-through recess in the distal body 314 under the distal end of the needle shaft 318, as indicated by the arrows in FIG. 36.

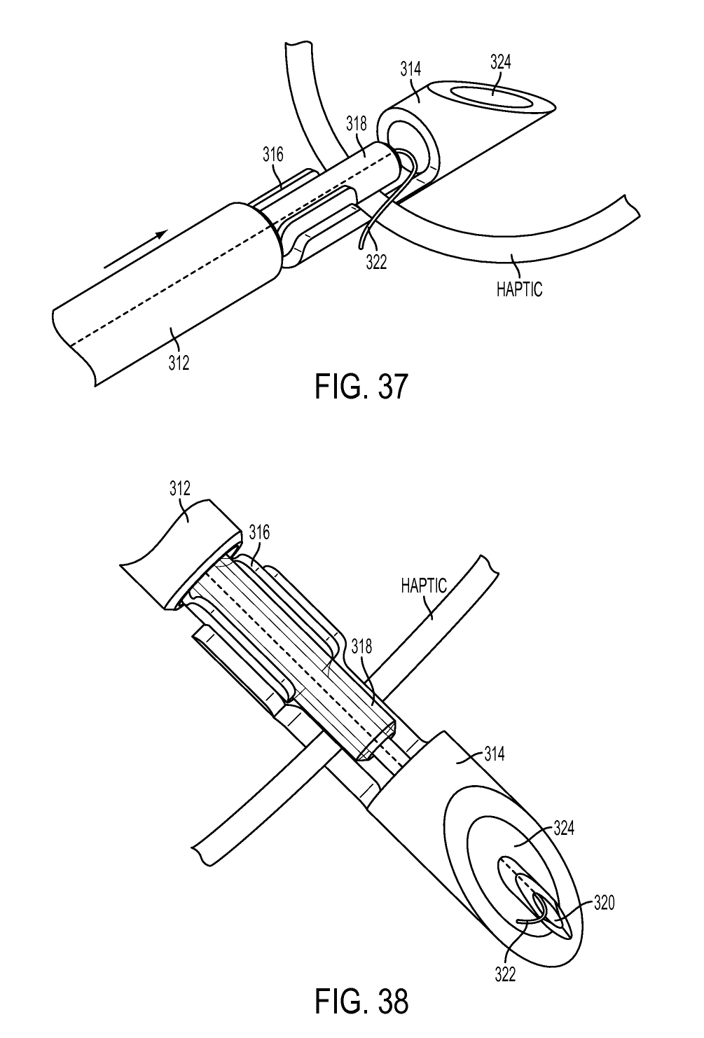

With the haptic in the pass-through recess of the distal body 314, the finger pads 306 can be released, which resiliently spread back apart and pull the actuator back distally, causing the shaft 312 to move back distally over the bent portion of the carrier 316, which causes the distal end of the carrier and the distal body 314 to close back against/toward the needle shaft 318, thereby capturing/clamping the haptic between the needle shaft 318 and the pass-through recess in the distal body, as shown in FIG. 37. With the haptic thus captured, the IOL is now fully controllable by the operator to push, pull, rotate, or otherwise reposition IOL as desired.

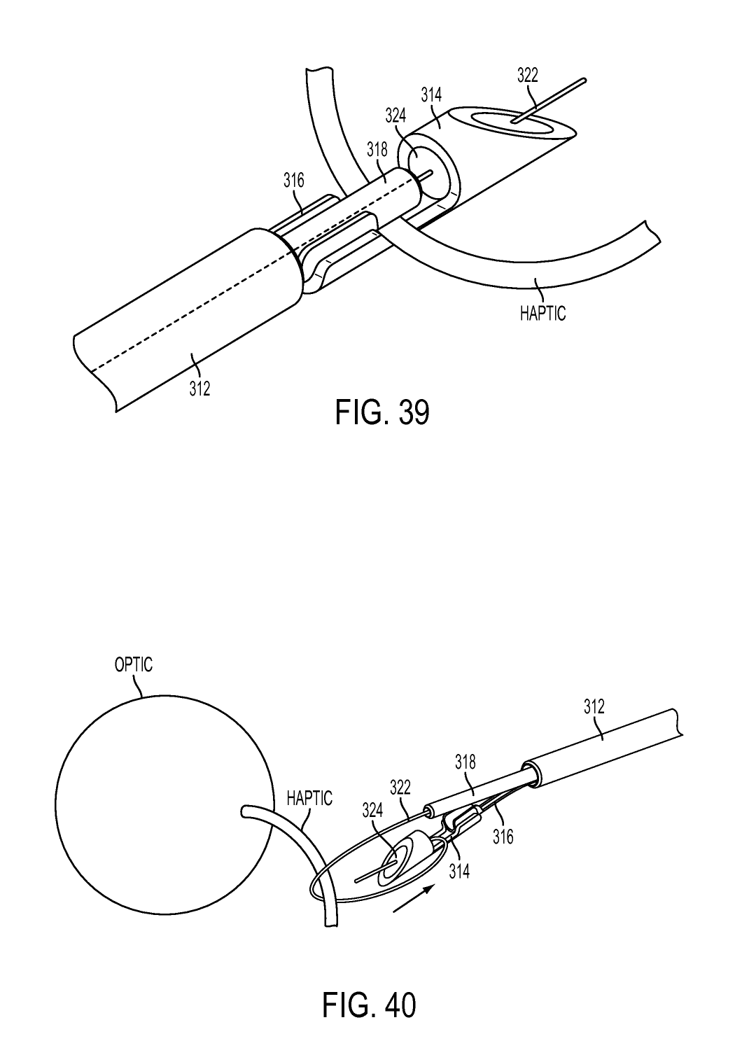

As shown in FIG. 34A, the device 300 can includes a plunger 321 projecting from the proximal end of the frame 302, or a similar lever or slider mechanism extending from the frame. Pushing the plunger 321 distally relative to the frame 302 causes the needle 320 to advance distally relative to the needle shaft 318 and distal body 324, as shown in FIG. 38. As the needle 320 advances distally, the needle passes through the plug 324 and pulls the suture 322 through the plug with it, as shown in FIG. 38. The plug 324 can comprise a resiliently deformable fill material, such as silicone. When the plunger 321 is released, it can be pushed back proximally relative to the frame 302 via a spring mechanism or other biasing mechanism (or can be manually moved back proximally), to cause the needle 320 to retract proximally back through the plug 324 and back into the needle shaft 318. However, the resiliency and surface friction of the plug material can shrink back around the suture 322 when the needle is retracted and the plug material can grip the suture and prevent the suture from being pulling back through the plug, leaving the suture extending from the needle through the plug and out of the distal end of the device, as shown in FIG. 39.

As shown in FIG. 40, the haptic can then be released by pinching the finger pads 306 again, opening the distal body 314 away from the needle shaft 318. As the device is retracted proximally from the haptic, the haptic moves laterally out of the pass-through in the distal body 314 and the haptic pulls the suture 322 out of the needle shaft while slack is provided to the suture, as shown in FIG. 40. The proximal end of the suture 322 can be retained within the device 300 proximal to the plug 324 and needle 320 in a manner that allows slack to be automatically or selectively added to the suture as the device is retracted away from the haptic.

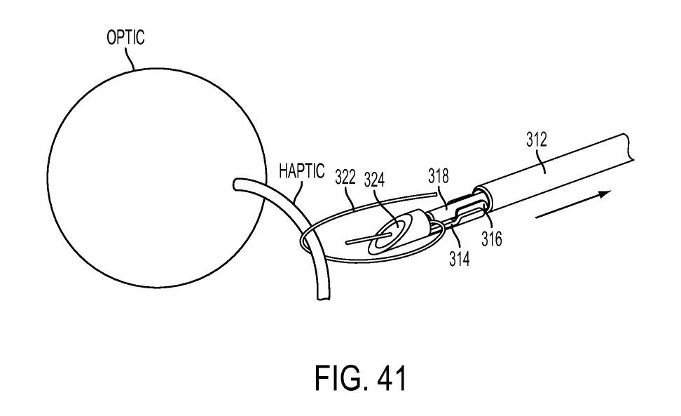

Once the haptic is out of the device 300, as shown in FIG. 40, the finger pads 306 can be released again to cause the shaft 312 to move distally over the bend in the carrier 316 and close the distal body 314 back against/toward to the needle shaft 318, as shown in FIG. 41. The device 300 can then be retracted out of the eye with the suture 322 remaining looped around the haptic as more slack is provided to the suture. Once the device 300 is out of the eye, the suture 322 can be released from the device and the two ends of the suture extending out of the eye can be manipulated to tension the haptic as desired, and then secured, such as by tying them together, optionally using a bolster against the outer surface of the scleral wall. In some cases, simply repositioning the haptic while it is clamped by the device 300 is sufficient to correct the positioning of the IOL, without employing a suture.

In some embodiments, a second MVR stab adjacent to the first can be created, and one end of the suture 322 can be pulled/passed through the second stab (such as by inserting the device 300 or another device through the second stab and gripping one of the suture ends and then pulling it out through the second stab. With one end of the suture passing out of the eye through each of the stabs, the suture ends can be tied together (or otherwise secured) over the sclera, such as by using a bolster.

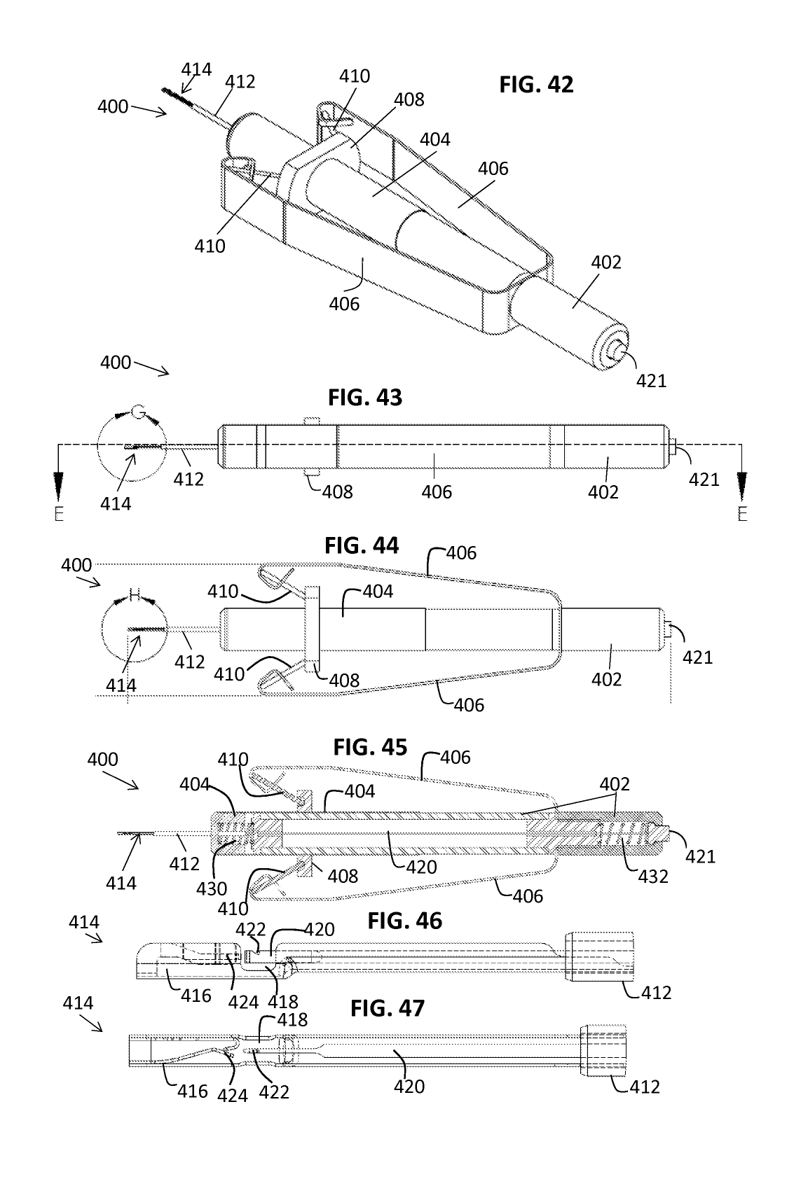

FIGS. 42-47 illustrate another single stab device 400 for repositioning and retaining an IOC. The device 400 includes a generally cylindrical or tubular frame 402, an actuator 404 mounted on the frame and adapted to slide longitudinally over the frame, flexible finger pads 406 having a fixed portion mounted on the frame, such as at a rear end, and free ends that can resiliently flex toward the frame and actuator when pinched together by an operator, a collar 408 mounted to the actuator, links 410 that couple the free ends of the finger pads to the collar, an introduction shaft 412 that extends from the distal end of the actuator for insertion into the eye, and a distal body 414 for puncturing the eye wall during insertion, capturing a haptic, and placing a suture around the haptic. As shown in FIGS. 46 and 47, the distal body 414 of the device 400 includes a first arm, or carrier, 416 and a second arm, or needle, 420 extending from within the distal end of the shaft 412. The needle 420 can carry a suture (not shown) threaded through or along the needle and held in a notch 422 at the end of the needle. The carrier 416 has a haptic pass-through recess 418 for below the distal end of the needle 420 for receiving and capturing the haptic. The clip has a discontinuity between the end of the needle 420 and the carrier 416 that can receive a haptic and can be closed to form a continuous, rigid engagement around the haptic.

In the unactuated configuration shown, with a suture mounted in the notch 422, the device 400 can be inserted through the scleral wall such that the shaft 412 is inserted across the scleral wall and the frame 402, finger portions 406, etc. are outside of the eye, either with or without a preliminary incision made in the scleral wall. For example, a preliminary MVR stab incision can be made and the device 400 can pass through the incision. Once inserted into the ciliary sulcus, the operator can pinch the finger pads 406 to cause the actuator 404 to move longitudinally relative to the frame 402, via the links 410 and collar 408. A spring 430 (FIG. 45) can provide a biasing force to return the actuator distally when the finger pads 406 are released. Longitudinal motion of the actuator 404 causes the a haptic receiving gap to open between the distal end of the needle 420 and the carrier 416. The device 400 can then be manipulated to cause a haptic to move into a haptic pass-through recess 418 in the carrier under the distal end of the needle 420.

With the haptic in the pass-through recess 418, the finger pads 406 can be released, which can resiliently spread back apart, causing the needle 420 and the carrier 416 to close around the haptic, thereby capturing/clamping the haptic. With the haptic thus captured, the IOL is now fully controllable by the operator to push, pull, rotate, or otherwise reposition IOL as desired.

As shown in FIG. 42, the device 400 can includes a plunger 421 projecting from the proximal end of the frame 402, or a similar lever or mechanism extending from the frame. Pushing the plunger 421 distally relative to the frame 402 causes the needle 420 to advance distally relative to the carrier 416. As the needle 420 advances distally, the needle passes through a suture grabber 424 at the distal end of the carrier and pulls the suture in the notch 422 through the suture grabber 424 along with it. The suture grabber can comprise any kind of gripping structure, such as two flanges with ends near each other that can deflect to allow the needle to pass and then clamp back around the suture. When the plunger 421 is released, it can be pushed back proximally relative to the frame 402 via a spring 432 in the frame, or can be manually pulled back proximally by the operator, to cause the needle 420 to retract proximally back through the suture grabber 424, leaving the suture extending over the haptic and secured in the suture grabber.

The haptic can then be released by pinching the finger pads 406 again to open the distal body 414. As the device 400 is retracted proximally from the haptic, the haptic moves laterally out of the pass-through 418 and the haptic pulls the suture out of the shaft 412 while slack is provided to the suture and the end of the suture remains mounted in the suture grabber 424. The proximal end of the suture can be retained within the device 400 proximal to the plug and needle in a manner that allows slack to be automatically or selectively added to the suture as the device is retracted away from the haptic.

Once the haptic is out of the device 400, the finger pads 406 can be released again to cause the distal body 414 to close again. The device 400 can then be retracted out of the eye with the suture remaining looped around the haptic as more slack is provided to the suture. Once the device 400 is out of the eye, the suture can be released from the device and the two ends of the suture extending out of the eye can be manipulated to tension the haptic as desired, and then secured, such as by tying them together, optionally using a bolster against the outer surface of the scleral wall.

The same or similar procedures can optionally be performed at an opposite side of the eye to reposition and/or retain the other haptic of the IOL.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. The term “comprises” means “includes without limitation.” The term “coupled” means physically linked and does not exclude intermediate elements between the coupled elements. The term “and/or” means any one or more of the elements listed. Thus, the term “A and/or B” means “A”, “B” or “A and B.”

The materials, methods, and examples disclosed herein are illustrative only and not intended to be limiting. Although methods and materials similar or equivalent to those described herein can be used in the practice or testing of the present technology, only certain suitable methods and materials are described herein. In case of conflict, the present specification, including terms, will control. In addition, the materials, methods, and devices are illustrative only and not intended to be limiting.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the following claims. We therefore claim all that comes within the scope of the following claims.

1. A method of repositioning an intraocular lens (IOL) previously implanted in the eye, comprising:
inserting an introduction instrument through a wall of the eye near the ciliary body and into the ciliary sulcus;
using the introduction instrument, positioning a rigid clip having two arms with a discontinuity between the two arms over a haptic of the previously implanted IOL;
closing the discontinuity between the two arms of the clip such that the clip forms a continuous rigid engagement around the haptic; and
repositioning the IOL within the eye by moving the engaged haptic with the introduction instrument.

2. The method of claim 1, further comprising:
inserting the introduction instrument through the wall of the eye into the ciliary sulcus at an opposite side of the previously implanted IOL relative to the initial insertion of the introduction instrument;
using the introduction instrument, positioning a rigid clip having a discontinuity over a second haptic of the IOL;
closing the discontinuity of the clip such that the clip forms a continuous engagement around the second haptic; and
repositioning the IOL to a desired position within the eye by moving the engaged second haptic of the IOL.

3. The method of claim 1, wherein closing the discontinuity of the clip comprises securely grasping the haptic with the clip.

4. The method of claim 1, wherein repositioning the IOL comprises moving one or more haptics of the IOL into the ciliary sulcus.

5. The method of claim 1, further comprising positioning a suture around the haptic using the clip and securing the suture to the eye wall to retain the haptic at a desired position within the eye.

6. The method of claim 1, wherein engaging the haptic with the clip comprises:
opening the discontinuity between the arms of the clip within the eye;
causing a non-end portion of the haptic to enter the open discontinuity such that the open discontinuity moves over the non-end portion of the haptic in a direction transverse to a longitudinal direction of the haptic; and
closing the the discontinuity between the arms of the clip to capture the non-end portion of the haptic in the clip.

7. The method of claim 6, further comprising:
with the haptic engaged within the clip, advancing a suture transversely over the haptic and retaining the suture to a distal end portion of the clip;
after the suture is retained to the distal end portion of the clip, opening the discontinuity in the clip; and
retracting the clip such that the haptic exits through the discontinuity and the suture becomes looped around the haptic.

8. The method of claim 7, further comprising:
after retracting the clip, closing the clip with the suture looped around the haptic;
pulling the closed clip and a portion of the suture out through the eye wall; and
securing the suture to the eye wall to tether the haptic to the eye wall.

9. The method of claim 7, wherein advancing the suture over the haptic and retaining the suture to the distal end portion of the clip comprises:
advancing a needle across the haptic and through a resiliently deformable plug in the distal end portion of the clip such that the needle carries the suture through the plug; and
retracting the needle from the plug such that the suture remains retained by the plug.

10. The method of claim 6, wherein opening the discontinuity between the arms of the clip comprises:
retracting an introduction sheath of the introduction instrument to uncover a pre-bent portion of an arm extending through the sheath, wherein the pre-bent portion is held in a straightened configuration when covered by the sheath, and wherein the pre-bent portion resiliently returns to a bent configuration when uncovered by the sheath.

11-17. (canceled)

18. A device for repositioning a previously implanted intraocular lens (IOL) in the eye, comprising:
a frame adapted to be held by a surgeon;
an elongated insertion sheath extending from the frame and adapted to be inserted through an eye wall;
an actuator coupled to the frame and adapted to control longitudinal movement of the insertion sheath relative to the frame;
an engagement portion that extends through the insertion sheath and comprises two rigid arms that are adapted to open apart from each other and close together to receive and capture a haptic of the previously implanted IOL between the arms;
wherein the actuator is operable to cause the insertion sheath to retract relative to the engagement portion such that the engagement portion resiliently opens to receive the haptic, and wherein the actuator is operable to causes the insertion sheath to advance relative to the engagement portion with the haptic received such that the engagement portion closes and captures the haptic.

19. The device of claim 18, wherein the engagement portion comprises a first arm and a second arm, the second arm having a pre-bent portion that causes an end portion of the second arm to resiliently move apart from the first arm when the pre-bent portion is uncovered by the insertion sheath.

20. The device of claim 19, wherein the end portion of the second arm comprises a recess or trough for receiving the haptic.

21. The device of claim 19, wherein the end portion of the second arm comprises a plug that is positioned distal to and in longitudinal alignment with distal end of the first arm when the engagement portion is closed.

22. The device of claim 21, wherein the first arm comprises a needle sheath and a needle that is longitudinally slidable within the needle sheath upon actuation of a needle controller coupled to the frame outside of the eye.

23. The device of claim 22, wherein actuation of the needle controller causes the needle to advance from the needle sheath and pierce through the plug, carrying a suture from the needle sheath through the plug.

24. The device of claim 23, wherein the plug is adapted to retain the suture after the needle is retracted back out of the plug and into the needle sheath.

25. The device of claim 18, wherein the actuator for controlling longitudinal movement of the insertion sheath comprises two flexible finger pads positioned on opposite sides of the frame, a collar mounted around the frame, and two links coupling the finger pads to the collar, wherein squeezing the finger pads toward the frame causes the collar to move longitudinally away from the eye wall relative to the frame and thereby retract the insertion sheath relative to the frame.

26-43. (canceled)

44. The device of claim 21, wherein the end portion of the second arm comprises a sharp tubular portion distal to the recess or trough for receiving the haptic, the sharp tubular portion is operable to be inserted through a wall of the eye or through an incision in the eye wall, and the plug is mounted within a lumen of the tubular portion.

45. The device of claim 22, wherein when the engagement portion is in an open position to receive the haptic, the end portion of the second arm extends longitudinally along an axis that forms an acute angle relative to an longitudinal axis of the needle sheath of the first arm, such that the plug is not aligned with the longitudinal axis of the needle sheath and a gap is formed between a distal end of the needle sheath and the end portion of the second arm such that the haptic can move laterally through the gap into the recess or trough in the second arm.
